(19) United States
(12) Patent Application Publication
(10) Pub. No.: US 2025/0222098 A1
(43) Pub. Date: Jul. 10, 2025

(54) Novel Peptides and Vaccines Capable of Eliciting Protective Immunity Against SARS-CoV-2

(71) Applicant: BIOMED PROTECTION, L.L.C., Galveston, TX (US)

(72) Inventors: VELJKO VELJKOVIC, Belgrade (RS); SLOBODAN PAESSLER, Galveston, TX (US)

(21) Appl. No.: 18/852,698

(22) PCT Filed: Apr. 3, 2023
(86) PCT No.: PCT/US2023/065286

§ 371 Date: Sep. 30, 2024

Related U.S. Application Data
(60) Provisional application No. 63/326,545, filed on Apr. 1, 2022.

(51) Int. Cl. A61K 39/215; A61K 39/00; A61P 31/14; C07K 16/10; C12N 7/00; G01N 33/68

(52) U.S. Cl. CPC A61K 39/215; A61P 31/14; C07K 16/1003; C12N 7/00; G01N 33/6854; A61K 2039/575; C12N 2770/20034; G01N 2333/165

(57) ABSTRACT

The invention generally relates to the development of a vaccine capable of providing broad protection against SARS-CoV-2 virus as well as the production of antibodies to treat the COVID-19 disease. More particularly, the invention related to the use of an informational spectrum method (ISM) to identify novel peptides sharing structural and informational homology with spike protein subunit 1 (SP1) from SARS-CoV-2, and vaccines comprising the SP1 multi-epitope peptide antigen that is capable of eliciting protective immunity against SARS-CoV-2. The SARS-CoV-2 vaccine can be used alone or in combination with other COVID-19 vaccines.

### CROSS-REFERENCE TO RELATED APPLICATIONS

This application is being filed on Apr. 3, 2023, as a PCT International Patent Application and claims the benefit of and priority to U.S. Provisional Patent Application Ser. No. 63/326,545, filed on Apr. 1, 2022, the entirety of which is incorporated herein by reference.

### BACKGROUND

The Coronavirus Disease 2019 (COVID-19) pandemic, caused by Severe Acute Respiratory Syndrome Coronavirus 2 (SARS-CoV-2), represents an unprecedented health threat resulting in over 1.1 million deaths globally and significant economic damage. See www.worldometers.info/coronavirus. There is a consensus that development of an effective and safe vaccine represents the best strategy to control COVID-19 pandemic, and consequently, this endeavor remains a top public health priority. Researchers and drug companies around the world are working hard to develop a COVID-19 vaccine, the spike glycoprotein (SP) of SARS-CoV-2 is the principal target for most these vaccine candidates. The GISAID database contains more than 1,600 SARS-CoV-2 strains with unique combinations of mutations in SP. See https://www.gisaid.org. Despite the presence of a CoV proof-reading function in viral replication, SARS-CoV still extensively mutates, which may impact current and future vaccine development. There is the concern that changes in the SP representing the principal antigenic determinant of SARS-CoV-2 will produce viruses that elude resistance elicited by existing vaccines.

Accordingly, the identification of novel antigens that are capable of eliciting an immune response against various mutants of SARS-CoV-2 viruses to provide cross-strain protection against different SARS-CoV-2 strains remains desirable.

### SUMMARY

The embodiments of the present application provide a method for identifying novel antigens possessing structural homology with spike glycoprotein 1 (SP1) from SARS-CoV-2 which is capable of eliciting an immune response against different strains of virus. Such antigens can be used as the basis for a COVID-19 vaccine, suitable for use alone or in combination with other vaccines against SARS-CoV-2.

In one aspect, a method of eliciting an immune response is provided by administering an immunologic composition comprising a peptide having an informational spectrum (IS) that overlaps with the IS of SP1 protein or fragment thereof, wherein the peptide is capable of eliciting an immune response against the SP1 protein or fragment thereof. The SP1 protein and the peptide may be homologous such that the peptide is suitable for use as an antigen to produce antibodies, preferably neutralizing antibodies that bind to the SP1 protein or fragment thereof.

In one aspect, a cross-spectrum (CS) based on the IS of the SP1 protein or fragment thereof (determined using an ISM approach as described herein) and the IS of the peptide may contain frequency components of F(0.257) and F(0.479). Additionally, the IS of the peptide may comprise F(0.255-0.259) and F(0.477-0.481).

In one aspect, a peptide may comprise 36 to 646 amino acids. More particularly, the peptide may comprise 15 to 65 amino acids.

In one aspect, the peptide has the sequence of SEQ ID NO: 1, which is:
LKYNENGTITDAVDCALDPLSETKCTLKSFTVEKGIYQTSNFRVQPTESIVRFPN
ITNLCPFGEVFNATRFASVY.
In one aspect, the peptide has the sequence of SEQ ID NO: 2, which is:
DITPCSFGGVSVITPGTNTSNQVAVLYQGVNCTEVPVAIHADQLTPTWRVYSTG
SNVFQTRAGCL.
In one aspect, the peptide has the sequence of SEQ ID NO: 3, which is:
FLLKYNENGTITDAVDCALDPLSETKCTLKSFTVEKGIYQTSNFRVQPTESIVRF
PNITNLCPFGEVFNATRFASVYAWNRKRISNCVA.
In one aspect, the peptide has the sequence of SEQ ID NO: 4, which is:
DHWADKYWRAAMSWHRWWPDYQPDYWHDYYYRWHQMQERQMNRWQER
WWHRQAYSSPQQTESWRIDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 5, which is:
DHWADKYWRAAMSWHRWWPDYQPDYWHDYYYRWHQMQERQMNRWQER
WWHRQAYSSPQLTPTWRVDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 6, which is:
DHWADKYWRAAMSWHRWWPDYQPDYWHDYYYRWHQMQERQMNRWQER
WWHRQAGSTPQLTPTWRVDKTHTCPPCPAPELLGGPSVFLFPPKPKDTLMISR
TPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 7, which is:
DHWADKYWRAAMSWHRWWPDYQPDYWHDYYYRWHQMQERQMNRWQER
WWHRQAYSSPQQTESWRI.
In one aspect, the peptide has the sequence of SEQ ID NO: 8, which is:
SWHRWWPDYQPDYWHDYYYRWHQMQERQMNRWQERWWHRQAYSSPQLT
PTWRV.
In one aspect, the peptide has the sequence of SEQ ID NO: 9, which is:
DHWADKYWRAAMSWHRWWPDYQPDYWHDYYYRWHQMQERQMNRWQER
WWHRQAGSTPQLTPTWRV.
In one aspect, the peptide has the sequence of SEQ ID NO: 10, which is:
FLLKYNENGTITDAVDCALDPLSETKCTLKSFTVEKGIYQTSNFRVQPTESIVRF
PNITNLCPFGSNNLDSKVGGNYNYLYRLFRKSNLKPFERDISTEIYQAGSTPCNG
VEGFNCYFPLQSYGFQPTNGVGYQPY.
In one aspect, the peptide has the sequence of SEQ ID NO: 11, which is:
FLLKYNENGTITDAVDCALDPLSETKCTLKSFTVEKGIYQTSNFRVQPTESIVRF
PNITNLCPFGEVENATRFASVYAWNRKRISNCVASNNLDSKVGGNYNYLYRLF
RKSNLKPFERDISTEIYQAGSTPCNGVEGENCYFPLQSYGFQPTNGVGYQPY.
In one aspect, the peptide has the sequence of SEQ ID NO: 12, which is:
DHWADKYWRAAMSWHRWWPDYQPDYWHDYYYRWHQMQERQMNRWQER
WWHRQAYSSPQLTPTWRVSNNLDSKVGGNYNYLYRLFRKSNLKPFERDISTEI
YQAGSTPCNGVEGENCYFPLQSYGFQPTNGVGYQPY.
In one aspect, the peptide has the sequence of SEQ ID NO: 13, which is:
DHWADKYWRAAMSWHRWWPDYQPDYWHDYYYRWHQMQERQMNRWQER
WWHRQAGSTPQLTPTWRVSNNLDSKVGGNYNYLYRLFRKSNLKPFERDISTEI
YQAGSTPCNGVEGFNCYFPLQSYGFQPTNGVGYQPY.
In one aspect, the peptide has the sequence of SEQ ID NO: 14, which is:
DHWADKYWRAAMSWHRWWPDYQPDYWHDYYYRWHQMQERQMNRWQER
WWHRQAYSSPQQTESWRIRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 15, which is:
DHWADKYWRAAMSWHRWWPDYQPDYWHDYYYRWHQMQERQMNRWQER
WWHRQAYSSPQLTPTWRVRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRVVVL
SFELLHAPATVCGPKKSTNLVKNK.
In one aspect, the peptide has the sequence of SEQ ID NO: 16, which is:
DHWADKYWRAAMSWHRWWPDYQPDYWHDYYYRWHQMQERQMNRWQER
WWHRQAGSTPQLTPTWRVRVQPTESIVRFPNITNLCPFGEVENATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 17, which is:
DKQADKWWDYATTWHDQQPDWQPDYQKDWYWRQHSTMERQTGDWSEDQ
QHDQYYRTHQMRERQRI.
In one aspect, the peptide has the sequence of SEQ ID NO: 18, which is:
DKQADKWWDYATTWHDQQPDWQPDYQKDWYWRQHSTMERQTGDWSEDQ
QHDQYYRTHQMRERQRISNNLDSKVGGNYNYLYRLFRKSNLKPFERDISTEIY
QAGSTPCNGVEGFNCYFPLQSYGFQPTNGVGYQPY.
In one aspect, the peptide has the sequence of SEQ ID NO: 19, which is:
DKQADKWWDYATTWHDQQPDWQPDYQKDWYWRQHSTMERQTGDWSEDQ
QHDQYYRTHQMRERQRIRVQPTESIVRFPNITNLCPFGEVENATRFASVYAWN
RKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLS
FELLHAPATVCGPKKSTNLVKNK.
In one aspect, the peptide has the sequence of SEQ ID NO: 20, which is:
DKQADKWWDYATTWHDQQPDWQPDYQKDWYWRQHSTMERQTGDWSEDQ
QHDQYYRTHQMRERQRISNNLDSKVGGNYNYLYRLFRKSNLKPFERDISTEIY
QAGSTPCNGVEGENCYFPLQSYGFQPTNGVGYQPYDKTHTCPPCPAPELLGGP
SVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
In one aspect, the peptide has the sequence of SEQ ID NO: 21, which is:
SQCVNDTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQ
PFLMDLEGKQGNFKNLREFVFKNIDGYFKIYSKHTPINLVRDLPQGFSALEPLV
DLPIGINITRFQTLLADHRSYLTPGDSSSGWTAGAAAYYVGYLQPRTFLLKYNE
NGTITDAVDCALDPLSETKCTLKSFTVEKGIYQTSNFRVQPTESIVRFPNITNLCP
FGEVFNATRFASV.
In one aspect, the peptide has the sequence of SEQ ID NO: 22, which is:
YAWNRKRISNCVADYSVLYNSASFSTFKCYGVSPTKLNDLCFTNVYAISFVIRG
DEVRQIAPGQTGKIADYNYKLPDDFTGCVIAWNSNNLDSKVGGNYNYLYRLF
RKSNLKPFERDISTEIYQAGSTPCNGVEGENCYFPLQSYGFQPTNGVGYQPYRV
VVLSFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQGVNC
TEVPVAIHADQLTPTWRVYSTGSNVFQTRAGCLIGAEHVNNSYECDIPIGAGIC
ASYQTQTNSPRRAR.
In one aspect, the peptide has the sequence of SEQ ID NO: 23, which is:
GKQGNFKNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTITDAV
DCALDPLSETKCTLKSFTVEKGIYQTSNFRVQPTESIVRFPNITNL.
In one aspect, the peptide has the sequence of SEQ ID NO: 24, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TLAVDCALDPLSETKCTLKSFTVEKGIYQTSNFRVQPTEVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYF.
In one aspect, the peptide has the sequence of SEQ ID NO: 25, which is:
PLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVI
TPGTNTSNQVAVLYQDVNCTEVPVAIHADQLTPTWRVYSTGSNVFQTRAGCLI
GAEHVNNSYE.
In one aspect, the peptide has the sequence of SEQ ID NO: 26, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TLAVDCALDPLSETKCTLKSFTVEKGIYQTSNFRVQPTIVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVLTES
NKKFLPFQQFGRDIADTTDAVRDPQTLEILDITPCSFGGVSVITPGTNTSNQVAV
LYQDVNCTEVPVAIHADQLTPTWRVYSTGSNVFQTRAGCLIGAEHVNNSYE.
In one aspect, the peptide has the sequence of SEQ ID NO: 27, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TDAVDCALDPLSETKCTLKSFTVEKGIYQTSNFRVQPTIVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVLTES
NKKFLPFQQFGRDIADTTDAVRDPQTLEILDITPCSFGGVSVITPGTNTSNQVAV
LYQDVNCTEVPVAIHADQLTPTWRVYSTGSNVFQTRAGCLIGAEHVNNSYE.
In one aspect, the peptide has the sequence of SEQ ID NO: 28, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TDAVDCALDPLSETKCTLKSFTVEKGIYQTSNFRVQPTEVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVLTES
NKKFLPFQQFGRDIADTTDAVRDPQTLEILDITPCSFGGVSVITPGTNTSNQVAV
LYQDVNCTEVPVAIHADQLTPTWRVYSTGSNVFQTRAGCLIGAEHVNNSYE.
In one aspect, the peptide has the sequence of SEQ ID NO: 29, which is:
VNLTTRTQDPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 30, which is:
VNLTTRTQLPPAYTNSFTRGVYYPDKVFRSSVLHSTQLLFLPFFSNVTWFHAIH
VSGTNGTKRFDNPVLPFNLGVYFASTEKSNIIRGWIFGTTLDSKTQSLLIVNNAT
NVVIKVCEFQFCNDPFLGVYYHKNNKSMESEFRVYSSANNCTFEYVSQPFLMD
LEGKQGNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENGTIT
DAVDCALDPLSETKCTLKSFTVEKGIYQTSNFRVQPTESIVRFPNITNLCPFGEV
FNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGG
NYNYLYRLFRKSNLKPFERDISTEIYQAGSTPCNGVEGENCYFPLQSYGFQPTN
GVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQV
AVLYQDVNCTEVPVAIHADQLTPTWRVYSTGSNVFQTRAGCLIGAEHVNNSY
E.
In one aspect, the peptide has the sequence of SEQ ID NO: 31, which is:
VNLTTRTQDPPAYTNSFTRGVYYPDKVFRSSVLHSTQDLFLPFFSNVTWFHAIH
VSGTNGTKRFDNPVLPFNLGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 32, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 33, which is:
VNLTLRTQLPPAYTNSFTRGVYYPDKVFRSSVLHSTQLLFLPFFSNVTWFHAIH
VSGTNGTKRFDNPVLPFNLGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLDSKVG
GNYNYLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 34, which is:
VNLTTRTQDPPAYTNSFTRGVYYPDKVFRSSVLHSTQDLFLPFFSNVTWFHAIH
VSGTNGTKRFDNPVLPFNLGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 35, which is:
LKYNENGTITDAVDCALDPLSETKCTLKSFTVEKGIYQTSNFRVQPTESIVRFPN
ITNLCPFGEVFNATRFASVY.
In one aspect, the peptide has the sequence of SEQ ID NO: 36, which is:
DITPCSFGGVSVITPGTNTSNQVAVLYQGVNCTEVPVAIHADQLTPTWRVYSTG
SNVFQTRAGCL.
In one aspect, the peptide has the sequence of SEQ ID NO: 37, which is:
FLLKYNENGTITDAVDCALDPLSETKCTLKSFTVEKGIYQTSNFRVQPTESIVRF
PNITNLCPFGEVFNATRFASVYAWNRKRISNCVA.
In one aspect, the peptide has the sequence of SEQ ID NO: 38, which is:
DHWADKYWRAAMSWHRWWPDYQPDYWHDYYYRWHQMQERQMNRWQER
WWHRQAYSSPQQTESWRIDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 39, which is:
DHWADKYWRAAMSWHRWWPDYQPDYWHDYYYRWHQMQERQMNRWQER
WWHRQAYSSPQLTPTWRVDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 40, which is:
DHWADKYWRAAMSWHRWWPDYQPDYWHDYYYRWHQMQERQMNRWQER
WWHRQAGSTPQLTPTWRVDKTHTCPPCPAPELLGGPSVFLFPPKPKDTLMISR
TPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 41, which is:
DHWADKYWRAAMSWHRWWPDYQPDYWHDYYYRWHQMQERQMNRWQER
WWHRQAYSSPQQTESWRI.
In one aspect, the peptide has the sequence of SEQ ID NO: 42, which is:
SWHRWWPDYQPDYWHDYYYRWHQMQERQMNRWQERWWHRQAYSSPQLT
PTWRV.
In one aspect, the peptide has the sequence of SEQ ID NO: 43, which is:
DHWADKYWRAAMSWHRWWPDYQPDYWHDYYYRWHQMQERQMNRWQER
WWHRQAGSTPQLTPTWRV.
In one aspect, the peptide has the sequence of SEQ ID NO: 44, which is:
FLLKYNENGTITDAVDCALDPLSETKCTLKSFTVEKGIYQTSNFRVQPTESIVRF
PNITNLCPFGSNNLDSKVGGNYNYLYRLFRKSNLKPFERDISTEIYQAGSTPCNG
VEGFNCYFPLQSYGFQPTNGVGYQPY.
In one aspect, the peptide has the sequence of SEQ ID NO: 45, which is:
FLLKYNENGTITDAVDCALDPLSETKCTLKSFTVEKGIYQTSNFRVQPTESIVRF
PNITNLCPFGEVFNATRFASVYAWNRKRISNCVASNNLDSKVGGNYNYLYRLF
RKSNLKPFERDISTEIYQAGSTPCNGVEGENCYFPLQSYGFQPTNGVGYQPY.
In one aspect, the peptide has the sequence of SEQ ID NO: 46, which is:
DHWADKYWRAAMSWHRWWPDYQPDYWHDYYYRWHQMQERQMNRWQER
WWHRQAYSSPQLTPTWRVSNNLDSKVGGNYNYLYRLFRKSNLKPFERDISTEI
YQAGSTPCNGVEGENCYFPLQSYGFQPTNGVGYQPY.
In one aspect, the peptide has the sequence of SEQ ID NO: 47, which is:
DHWADKYWRAAMSWHRWWPDYQPDYWHDYYYRWHQMQERQMNRWQER
WWHRQAGSTPQLTPTWRVSNNLDSKVGGNYNYLYRLFRKSNLKPFERDISTEI
YQAGSTPCNGVEGENCYFPLQSYGFQPTNGVGYQPY.
In one aspect, the peptide has the sequence of SEQ ID NO: 48, which is:
DHWADKYWRAAMSWHRWWPDYQPDYWHDYYYRWHQMQERQMNRWQER
WWHRQAYSSPQQTESWRIRVQPTESIVRFPNITNLCPFGEVFNATRFASVYAW
NRKRISNCVADYSVLYNSASFSTFKCYGVSPTKLNDLCFTNVY ADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 49, which is:
DHWADKYWRAAMSWHRWWPDYQPDYWHDYYYRWHQMQERQMNRWQER
WWHRQAYSSPQLTPTWRVRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 50, which is:
DHWADKYWRAAMSWHRWWPDYQPDYWHDYYYRWHQMQERQMNRWQER
WWHRQAGSTPQLTPTWRVRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 51, which is:
DKQADKWWDYATTWHDQQPDWQPDYQKDWYWRQHSTMERQTGDWSEDQ
QHDQYYRTHQMRERQRI.
In one aspect, the peptide has the sequence of SEQ ID NO: 52, which is:
DKQADKWWDYATTWHDQQPDWQPDYQKDWYWRQHSTMERQTGDWSEDQ
QHDQYYRTHQMRERQRISNNLDSKVGGNYNYLYRLFRKSNLKPFERDISTEIY
QAGSTPCNGVEGENCYFPLQSYGFQPTNGVGYQPY.
In one aspect, the peptide has the sequence of SEQ ID NO: 53, which is:
DKQADKWWDYATTWHDQQPDWQPDYQKDWYWRQHSTMERQTGDWSEDQ
QHDQYYRTHQMRERQRIRVQPTESIVRFPNITNLCPFGEVENATRFASVYAWN
RKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLS
FELLHAPATVCGPKKSTNLVKNK.
In one aspect, the peptide has the sequence of SEQ ID NO: 54, which is:
DKQADKWWDYATTWHDQQPDWQPDYQKDWYWRQHSTMER
QTGDWSEDQQHDQYYRTHQMRERQRISNNLDSKVGGNYNYLYRLFRKSNLK
PFERDISTEIYQAGSTPCNGVEGENCYFPLQSYGFQPTNGVGYQPYDKTHTCPP
CPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQK
SLSLSPGK.
In one aspect, the peptide has the sequence of SEQ ID NO: 55, which is:
SQCVNDTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQ
PFLMDLEGKQGNFKNLREFVFKNIDGYFKIYSKHTPINLVRDLPQGFSALEPLV
DLPIGINITRFQTLLADHRSYLTPGDSSSGWTAGAAAYYVGYLQPRTFLLKYNE
NGTITDAVDCALDPLSETKCTLKSFTVEKGIYQTSNFRVQPTESIVRFPNITNLCP
FGEVFNATRFASVYAWNRKRISNCVADYSVLYNSASFSTFKCYGVSPTKLNDL
CFTNVYAISFVIRGDEVRQIAPGQTGKIADYNYKLPDDFTGCVIAWNSNNLDSK
VGGNYNYLYRLFRKSNLKPFERDISTEIYQAGSTPCNGVEGENCYFPLQSYGFQ
PTNGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITPGTNTSN
QVAVLYQGVNCTEVPVAIHADQLTPTWRVYSTGSNVFQTRAGCLIGAEHVNN
SYECDIPIGAGICASYQTQTNSPRRAR.
In one aspect, the peptide has the sequence of SEQ ID NO: 56, which is:
GKQGNFKNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTITDAV
DCALDPLSETKCTLKSFTVEKGIYQTSNFRVQPTESIVRFPNITNL.
In one aspect, the peptide has the sequence of SEQ ID NO: 57, which is:
GKQGNFKNLREFVFKNIDGYFKIYSKHTPINLDRILPQGFSALEPLVDLPIGINIT
RFQTLLADHRSYLTPGDSSSGWTAGAAAYYDGYLQPRTFLLKYNENGTITDAV
DCALDPLSETKCTLKSFTVEKGIYQTSNFRVQPTESIVRFPNITNL.

In one aspect, the peptide of the immunologic composition is suitable for use as an antigen in a vaccine. The peptide may be used alone or in combination with one or more other SARS-CoV-2 antigens. The resulting immunologic composition may provide protection against one or more virus strains.

In another aspect, the immunologic composition may further comprise a pharmaceutically acceptable carrier and/or excipient and/or adjuvant. Additionally, the immunologic composition may further comprise another antigen obtained from SARS-Cov-2 (e.g., ORF3b, ORF6, ORF8).

Further aspects provide for a method for producing the immunologic composition comprising (i) obtaining the peptide, and (ii) admixing the peptide with a pharmaceutically acceptable carrier and/or excipient and/or adjuvant.

Additional aspects contemplate a peptide that (i) comprises an amino acid sequence whose informational spectrum (IS) contains a frequency components F(0.257) and F(0.479), and (ii) produces antibodies that bind to a SP1 protein of SARS-CoV-2. The antibodies produced are preferably neutralizing antibodies. The peptide may be suitable for use as an antigen in an immunologic composition.

Certain aspects further encompass a method of using at least one peptide as an immunogen in order to generate antibodies, e.g., neutralizing antibodies that specifically bind to SP1 protein.

Additionally, certain aspects contemplate an isolated nucleic acid encoding the peptide: a vector containing the nucleic acid; and an isolated cell containing the vector as well as an antibody or antigen binding fragment thereof capable of binding to the peptide.

One aspect contemplates a therapeutic method or diagnostic method using the peptide or the antibody or antigen binding fragment thereof that binds to the peptide. For example, the peptide or the antibody or antigen binding fragment thereof that binds to the peptide are suitable for use a variety of diagnostic methods, including but not limited to, immunoassay's such as ELISA, ELISPOT, enzyme multiplied immunoassay technique, radioimmunoassay, and immunofluorescence; immunoblotting assays (such as a Western blot); immunoprecipitation assays such as chromatin immunoprecipitation (ChIP), immunodiffusion, and immunoelectrophoresis; immunocytochemistry; and immunohistochemistry. These diagnostic methods (and other similar methods) involve applying the peptide or the antibody or antigen binding fragment thereof that binds to the peptide to a sample (e.g., serum or mucosal) collected from a patient believed to have SARS-CoV-2 in order to detect and characterize the infection.

Another aspect encompasses a method for identifying a peptide that elicits an immune response against SP1 protein or fragment thereof. The method comprises (1) (i) obtaining an amino acid sequence of SP1 protein or fragment thereof; (ii) assigning an electron-ion interaction potential (EIIP) index value to each amino acid residue contained in the amino acid sequence of the SP1 protein or fragment; (iii) subjecting the resultant EIIP index values to discrete Fourier transformation (DFT); (iv) generating an informational spectrum (IS) of the SP1 protein or fragment based on the EIIP index values; (2) (i) obtaining an amino acid sequence of a peptide or peptides; (ii) assigning an EIIP index value to each amino acid residue contained in the amino acid sequence of the peptide or peptides; (iii) subjecting the resultant EIIP index values to discrete Fourier transformation (DFT); (iv) generating an informational spectrum (IS) of each peptide based on the EIIP index values; (3) comparing the IS of the SP1 protein or fragment generated in (1) (iv) to the IS of the peptide or peptides generated in (2) (iv); and (4) identifying the peptide or peptides whose IS overlap with the IS of the SP1 protein or fragment, and based thereon identifying the peptide or peptides as one being capable of eliciting an immune response against the SP1 protein or fragment.

The method may further include synthesizing at least one of the identified peptides and, optionally, assessing its immunogenicity or ability to generate antibodies, preferably neutralizing antibodies, that specifically bind to said SP1 protein. Additionally, the method may further include producing an immunologic composition comprising at least one of the identified peptides.

A cross-spectrum (CS) based on the IS of the SP1 protein or fragment thereof (determined using an ISM approach as described herein) and the IS of the peptide may contain a frequency components of F(0.257) and F(0.479). Additionally, the IS of the peptide may comprise F(0.255-0.259) and F(0.477-0.481). The peptide may comprise 64 to 545 amino acids. More particularly, the peptide may comprise 15 to 50 amino acids.

The peptide identified by the disclosed methods may be used in the preparation of an immunologic composition, which itself may contain the peptide alone or in combination with one or more additional SARS-CoV-2 vaccine antigens. The resulting immunologic composition containing the peptide may provide protection against one or more SARS-CoV-2 strains. Accordingly, certain aspects also contemplate a method for the treatment or prevention of SARS-CoV-2 virus infection in a subject, comprising administering a therapeutically effective amount of the immunologic composition or a therapeutically effective amount of the peptide or a therapeutically effective amount of the antibody or antigen binding fragment thereof to the subject in need thereof, preferably the treated subject is a human, such that the immunologic composition or the peptide or the antibody or antigen binding fragment thereof treats or prevents SARS-CoV-2 infection in the subject. The subject can also be a laboratory animal (e.g., mouse, rat, guinea pig, non-human primate), livestock (e.g., swine, avian), or a domestic animal (e.g., canine).

### BRIEF DESCRIPTION OF THE DRAWINGS

FIG. 1. shows the cross-spectrum (CS) of SP1 proteins from SARS-CoV-2, MERS and the ACE2 receptor. The abscissa represents frequencies and the ordinate represents the signal-to-noise ratio (S/N).

FIG. 2A shows a comparison of the consensus IS of SP1 proteins from SARS-Cov-1. The abscissa represents frequencies and the ordinate represents the signal-to-noise ratio (S/N).

FIG. 2B shows a comparison of the consensus IS of SP1 proteins from SARS-CoV-2. The abscissa represents frequencies and the ordinate represents the signal-to-noise ratio (S/N).

FIG. 3 shows the design of the SARS-CoV-2 challenge in hamsters.

FIG. 4A shows the body weight change results of the SARS-CoV-2 challenge in hamsters.

FIG. 4B shows the body temperature change results of the SARS-CoV-2 challenge in hamsters.

### DETAILED DESCRIPTION

Aspects of the present disclosure provide a novel approach to the identification of novel peptides whose Electron-Ion Interaction Potential (EIIP) structure, but not necessarily sequence, mimic conserved SARS-CoV-2 antigenic targets and, thus, can be used to elicit an immune response against SARS-CoV-2 virus. The EIIP structure is unrelated to the linear amino acid sequence (primary structure) and peptide folding motifs (e.g., secondary and tertiary structure). In particular, the peptides share EIIP homology with SP1 protein that can be derived from SARS-CoV-2 and, thus, can be used as an antigen (e.g., in a vaccine). As a result of the EIIP structural similarity between the identified peptide and the natural SP1 protein, antibodies provided against the peptide are also capable of recognizing the natural SP1 proteins. Accordingly, peptides whose structure mimics that of SP1 protein, can be used to elicit an immune response against the several different SP1 proteins and, thus, provide cross-protective immunity against several different SARS-CoV-2 strains. Table 1 shows the EIIP values for 20 amino acids that comprise peptides.

TABLE 1
EIIP Values for 20 Amino Acids

| Amino Acid | EIIP Value |
|---|---|
| Leucine (L) | 0.0000 |
| Isoleucine (I) | 0.0000 |
| Asparagine (B) | 0.0036 |
| Glycine (G) | 0.0050 |
| Valine (V) | 0.0057 |
| Glutamic Acid (Z) | 0.0058 |
| Proline (P) | 0.0198 |
| Histidine (H) | 0.0242 |
| Lysine (L) | 0.0371 |
| Alanine (A) | 0.0373 |
| Tyrosine (Y) | 0.0516 |
| Tryptophan (W) | 0.0548 |
| Glutamine (Q) | 0.0761 |
| Methionine (M) | 0.0823 |
| Serine (S) | 0.0829 |
| Cysteine (C) | 0.0829 |
| Threonine (T) | 0.0941 |
| Phenylalanine (F) | 0.0946 |
| Arginine (R) | 0.0959 |
| Aspartic Acid (D) | 0.1263 |

This disclosure generally relates to the development of a universal SARS-CoV-2 vaccine capable of providing broad protection against different strains of the virus. More particularly, the aspects of this disclosure are related to the use of an informational spectrum method (ISM) to identify novel peptides sharing structural homology with SP1 from SARS-CoV-2, and vaccines comprising the SP1 multi-epitope peptide antigen that is capable of eliciting cross-protective immunity against several different SARS-CoV-2 strains. The universal COVID-19 vaccine can be used alone or in combination with other SARS-Cov-2 vaccines.

Large clinical trials of four vaccine candidates are showing remarkable promise, with three exceeding 90% efficacy—an unexpectedly high rate—according to results released so far. One in particular has shown promise in older adults, a demographic that is particularly vulnerable to SARS-CoV-2, which also tends to have a muted response to vaccines. Early studies had shown that these candidate vaccines could stimulate an immune response. The latest trials show that this immune response can protect people against COVID-19—a major achievement.

However, vaccine development is fraught with possibilities for failure, and even the most ardent optimist might not have expected to have a highly effective vaccine against a new virus less than a year after its genome was sequenced. Despite the promising results obtained with the current vaccine candidates, some future problems may exist. The SARS-CoV-2 has been shown to mutate and could start to extensively mutate under the immune pressure which will caused by the mass vaccination. Therefore there remains a need for the development of an universal COVID-19 vaccine which is resistant or less sensitive to SARS-Cov-2 mutations. The development of such a universal COVID-19 vaccines relies, in large part, on the utilization of highly conserved antigenic targets. However, based on the experience with flu vaccines, conserved antigen epitopes are usually less exposed to the host immune system, and as such, are weakly immunogenic. Therefore, it is desirable to develop a novel approach to the identification of novel antigens having conserved properties which are capable of eliciting cross-protective immunity against different SARS-CoV-2 strains.

Aspects of the present disclosure use an ISM approach, rather than traditional neutralization assays, to identify structural characteristics of SARS-CoV-2 peptides and, based thereon, obtain novel peptide antigens having a spectral profile overlapping, at least in part, with the spectral profile of the SARS-CoV-2 peptide(s). The broad neutralization capacity against different SARS-CoV-2 strains, combined with the flexibility in the primary structure of the identified novel antigen(s) (i.e., the peptide(s) share structural homology, but not necessarily a linear amino acid sequence, with the SARS-CoV-2 peptide(s)), provide broad protectivity across different viral strains and, thus, provide an universal COVID-19 vaccine. See Veljkovic et al., Use of the informational spectrum methodology for rapid biological analysis of the novel coronavirus 2019-cCOV: prediction of potential receptor, natural reservoir, tropism and therapeutic/vaccine target. F1000 Research, (2021). 9 (52): 1-15, which is herein incorporated by reference in its entirety. Accordingly, these peptides as well as the immunologic compositions comprising them, can be used individually, in combination with one another, or in combination with other SARS-CoV-2 vaccine antigens.

In the ISM approach, sequences (protein or nucleotide) are transformed into signals by assigning a numerical value to each element (amino acid or nucleotide). These values correspond to the electron-ion interaction potential (EIIP), which determines electronic properties of amino acids and nucleotides. The signal obtained is then decomposed into a periodical function by Fourier transformation, resulting in a series of frequencies (F) and their amplitudes (A). The obtained frequencies correspond to the distribution of structural motifs with defined physico-chemical characteristics that are responsible for the biological function of the sequence. In other words, the peak frequencies of IS of a protein sequence reflect its biological or biochemical functions. See Veljkovic et al., Application of the EIIP/ISM bioinformatics concept in development of new drugs. Curr. Med. Chem. (2007) 14:133-135, which is herein incorporated by reference in its entirety. When comparing proteins that share the same biological or biochemical function(s), this technique allows the detection of code/frequency pairs that are specific for their common biological properties. The method is insensitive to the location of the motifs and, thus, does not require previous alignment of the sequences.

More particularly, it is generally believed that the number of valence electrons and the EIIP representing the main energy term of valence electrons are essential physical parameters determining the long-range properties of biological molecules. EIIP for organic molecules can be determined by the following simple equation derived from the “general model pseudopotential”:

$$W = 0.25 {Z}^{*} sin ( 1.04 {pZ}^{*} ) / 2 π$$

where Z* is the average quasivalence number (AQVN) determined by

Z*=ΣmniZi/N

And wherein Zi is the valence number of the i-th atomic component, ni is the number of atoms of the i-th component, m is the number of atomic components in the molecule, and N is the total number of atoms.

The EIIP values calculated according to equations (1) and (2) are in Rydbergs (Ry). The strong connection between EIIP and AQVN of organic molecules and their biological activity has previously demonstrated, e.g., in the context of mutagenicity, carcinogenicity, toxicity, antibiotic activity, and cytostatic activity.

A sequence of N residues is represented as a linear array of N terms, with each term given a weight. The weight assigned to a residue is EIIP, determining electronic properties of amino acids and nucleotides, which are responsible for their intermolecular interactions. In this way the alphabetic code of protein or nucleotide sequence is transformed into a sequence of numbers. The obtained numerical sequence, representing the primary structure of protein, is then subjected to a discrete Fourier transformation, which is defined as follows:

$$X ( n ) = \sum x ( m ) {e}^{j ( 2 / N ) nm} , n is 1 , 2 , … , N / 2$$

where x(m) is the m-th member of a given numerical series, N is the total number of points in this series, and X(n) are discrete Fourier transformation coefficients.

These coefficients describe the amplitude, phase and frequency of sinusoids, which comprise the original signal. The absolute value of complex discrete Fourier transformation defines the amplitude spectrum and the phase spectrum. The complete information about the original sequence is contained in both spectral functions.

In this way, sequences are analyzed as discrete signals. It is assumed that their points are equidistant with the distance d is 1. The maximal frequency in a spectrum defined in this way is F is ½d is 0.5. The frequency range is independent of the total number of points in the sequence. The total number of points in a sequence influences only the resolution of the spectrum. The resolution of the N-point sequence is 1/n. The n-th point in the spectral function corresponds to a frequency f (n) is of is n/N. Thus, the initial information defined by the sequence of amino acids can now be presented in the form of the informational spectrum (IS), representing the series of frequencies and their amplitudes.

The IS frequencies correspond to distribution of structural motifs with defined physicochemical properties determining a biological function of a protein. When comparing proteins, which share the same biological or biochemical function, the ISM technique allows detection of code/frequency pairs which are specific for their common biological properties, or which correlate with their specific interaction. This common informational characteristic of sequences is determined by a cross-spectrum (CS). A CS of N spectra is obtained by the following equation:

C(j)=πS(i,j)

where π(i,j) is the j-th element of the i-th power spectrum and C(j) is the j-th element of CS.

Thus, CS is the Fourier transform of the correlation function for the spectrum. In this way, any spectral component (frequency) not present in all compared informational spectra is eliminated. Peak frequencies in CS are common frequency components for the analyzed sequences. A measure of similarity for each peak is the signal-to-noise ratio (S/N), which represents a ratio between signal intensity at one particular IS frequency and the main value of the whole spectrum. If one calculates the CS for a group of proteins, which have different primary structures, and finds strictly defined peak frequencies, it means that primary structures of the analyzed proteins encode the same information. It has been demonstrated that: 1) such a peak exists only for the group of proteins with the same biological function: 2) no significant peak exists for biologically unrelated proteins: 3) peak frequencies are different for different biological functions. Furthermore, it has been shown that the proteins and their targets (ligand/receptor, antibody/antigen, etc.) have the same characteristic frequency(ies) in common. Thus, it can be postulated that IS frequencies characterize not only the general function but also recognition and interaction between a particular protein and its target. Once the characteristic frequency for a particular protein function/interaction is identified, it is possible then to utilize the ISM approach to predict the amino acids in the sequence, which essentially contribute to this frequency and are likely to be crucial for the observed function.

The calculation of the IS and CS of SP1 amino acid sequences allow for the identification of conserved domains, e.g., structural properties, that likely play a role in the interaction of the viral SP1 protein with the cellular receptor(s).

In some aspects of the present disclosure, ISM was applied to identify the spectral properties of SP1 protein from individual SARS-CoV-2 strains and, further, to identify shared spectral properties of the different HA proteins such that a “common epitope” was identified.

Using this information, peptides that differ in amino acid sequence from the naturally-occurring SARS-CoV-2 peptides but possess the same or similar structural features as the viral protein(s) that are important for interacting with the host cell have identified. As such, these peptides can be used as a novel SARS-Cov-2 antigen(s), e.g., capable of eliciting an immune response against the SP1 protein or fragment thereof, e.g., suitable for use as an antigen to produce antibodies (preferably, neutralizing antibodies) that bind to the SP1 protein or fragment thereof. Additionally, because the peptides contain structural features that shared among viral SP1 proteins from different SARS-CoV-2 strains, the peptides effectively possess a multi-epitope that may be capable of eliciting immunity against several different SARS-CoV-2 mutants and, thus, provide cross-protective or universal immunity.

The peptides described herein can be administered by different methods, e.g., intranasally, or through parenteral administration, such as through sub-cutaneous injection, intra-muscular injection, intravenous injection, intraperitoneal injection, or intradermal injection. The peptides can be used individually or in combination. Additionally, the peptide may be administered alone or as part of a composition that further comprises one or more pharmaceutically acceptable carriers, the proportion of which is determined by the solubility and chemical nature of the peptide, chosen route of administration and standard biological administration. Because inventive peptides may target proteins on the surfaces of the virus and/or the cell, to ensure efficacy, the carrier in such formulations optionally are free or substantially free (e.g., at least 90, 95, 98, or 99 wt %) of proteins that bind to the peptides.

Suitable pharmaceutically acceptable carriers for the compositions containing the peptides are described in the standard pharmaceutical texts. See, e.g., Remington's Pharmaceutical Sciences, 18th Ed., Mack Publishing Company, Easton, Pa. (1990). Specific non-limiting examples of suitable pharmaceutically acceptable carriers include water, saline, dextrose, glycerol, ethanol, or the like and combinations thereof. In addition, if desired, the composition can further contain minor amounts of auxiliary substances such as wetting or emulsifying agents, pH buffering agents that enhance the antiviral effectiveness of the composition.

The present peptides can also be provided as pharmaceutically acceptable salts. A “pharmaceutically acceptable salt” includes a salt with an inorganic base, organic base, inorganic acid, organic acid, basic amino acid, or acidic amino acid. As salts of inorganic bases, the disclosure includes, for example, alkali metals such as sodium or potassium: alkaline earth metals such as calcium and magnesium or aluminum; and ammonia. As salts of organic bases, the disclosure includes, for example, trimethylamine, triethylamine, pyridine, picoline, ethanolamine, diethanolamine, and triethanolamine. As salts of inorganic acids, the instant disclosure includes, for example, hydrochloric acid, hydroboric acid, nitric acid, sulfuric acid, and phosphoric acid. As salts of organic acids, the instant disclosure includes, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. As salts of basic amino acids, the instant disclosure includes, for example, arginine, lysine and ornithine. Acidic amino acids include, for example, aspartic acid and glutamic acid.

Depending on the route of administration, the composition may take the form of a solution, suspension, tablet, pill, capsule, sustained release formulation, powder, cream, lotion, emulsion, or the like.

For topical administration, the peptide can be formulated into a composition containing an effective amount of the peptide, typically 0.01 or 0.1 to 10%, of the peptide. Such compositions are typically in the form of a solution, cream, lotion, or emulsion.

For parenteral administration, the peptides of the present disclosure may be administered by intravenous, subcutaneous, intramuscular, intraperitoneal, or intradermal injection, alone or in compositions further comprising pharmaceutically accepted carriers. For administration by injection, it is preferred to use the peptide in a solution in a sterile aqueous vehicle which may also contain other solutes such as buffers or preservatives as well as sufficient quantities of pharmaceutically acceptable salts or of glucose to make the solution isotonic. The peptides of the present disclosure can be obtained in the form of therapeutically acceptable salts that are well-known in the art.

The peptides of the present disclosure may be used for treating viral infections of the respiratory tract. Thus, in one aspect, the peptides can also be delivered locally to the respiratory system, for example to the nose, sinus cavities, sinus membranes or lungs. The peptide(s), or pharmaceutical compositions containing one or more peptides, can be delivered to the respiratory system in any suitable manner, such as by inhalation via the mouth or intranasally. The present compositions can be dispensed as a powdered or liquid nasal spray, suspension, nose drops, a gel or ointment, through a tube or catheter, by syringe, by packtail, by pledget, or by submucosal infusion. The peptides may be conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the peptide and a suitable powder base such as lactose or starch. Examples of intranasal formulations and methods of administration can be found in PCT publications WO 01/41782, WO 00/33813, and U.S. Pat. Nos. 6,180,603:6,313,093; and 5,624,898. A propellant for an aerosol formulation may include compressed air, nitrogen, carbon dioxide, or a hydrocarbon based low boiling solvent. The peptide or peptides of the present disclosure can be conveniently delivered in the form of an aerosol spray presentation from a nebulizer or the like. In some aspects, the active ingredients (i.e., peptides) are suitably micronized so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns. In one embodiment, one or more of the peptides are packaged into a device that can deliver a predetermined, and generally effective, amount of the peptide via inhalation, for example a nasal spray or inhaler.

Some preferred embodiments utilize specific DNA vaccines and methods of providing protective immunity to vertebrates, particularly humans, against SARS-CoV-2 virus. “Protective immunity” conferred by the method of the invention can elicit humoral and/or cell-mediated immune responses to SARS-CoV-2 virus, but more importantly interferes with the activity, spread, or function of SARS-CoV-2 virus after vaccination. The DNA vaccines of embodiments of the invention are transcription units containing DNA encoding the peptides or peptides described herein. Some preferred embodiments include the method of administrating a DNA vaccine to an individual in whom protective immunization is desired.

Some preferred embodiments of the invention feature a DNA vaccine containing one or more peptides described herein or peptide component DNA transcription unit (i.e., an isolated nucleotide sequence encoding the desired peptide or peptide described herein). The nucleotide sequence is operably linked to transcriptional and translational regulatory sequences for expression of the peptide in a cell of a vertebrate. Preferably, the nucleotide sequence encoding an peptides or combinations of the peptides described herein are contained in a plasmid vector.

While preferred embodiments of the invention utilize DNA vaccines, any method of immunization can be used, as the humoral (antibody) immune response is the primary means of SARS-CoV-2 virus inactivation. Methods of immunization include, but are not limited to, recombinant protein vaccines, killed bacterial vaccines, vaccines purified from bacterial cultures, reassortment vaccines, vaccines expressed in vivo by a bacterial or viral vector, isolated bacterial cell walls, toxoids, killed cell toxoid preparations and mRNA vaccines.

Unless otherwise noted, the terms “classical” or “conventional” vaccines refer to the above vaccination methods and other vaccination methods that are well tested and understood by those of moderate skill in the art.

The term “immune response” refers herein to a cytotoxic T cells response or increased serum levels of antibodies to an peptide, or to the presence of neutralizing antibodies to the SARS-CoV-2 virus. The terms “protection” or “protective immunity” refers herein to the ability of the serum antibodies and cytotoxic T cell response induced during immunization to protect (partially or totally) against infection and/or disease caused by an infectious agent, such as the SARS-CoV-2 virus; the above terms also refer to the ability of the vaccines to produce an immune response that will inhibit, impair and/or inactivate the SARS-CoV-2 virus. Unless otherwise noted, the term's “inhibition” or “inactivation” refers to the inactivation of the respective SARS-CoV-2 virus being discussed by some part of the immune response, most likely the antibody response. That is, a vertebrate immunized by the DNA vaccines or conventional vaccines of the invention will be treatable with antibiotics once infected by a SARS-CoV-2 virus if not protected from the respective viral infection.

The term “promoter sequence” herein refers to a minimal sequence sufficient to direct transcription. Enhancer sequences influence promoter-dependent gene expression and may be located in the 5′ or 3′ regions of the native gene. Expression is constitutive or inducible by external signals or agents. Optionally, expression is cell-type specific, tissue-specific, or species specific.

By the term “transcriptional and translational regulatory sequences” is meant nucleotide sequences positioned adjacent to a DNA coding sequence which direct transcription or translation of a coding sequence. The regulatory nucleotide sequences include any sequences that promote sufficient expression of a desired coding sequence and presentation of the protein product to the inoculated vertebrate's immune system such that protective immunity is provided.

By the term “operably linked to transcriptional and translational regulatory sequences” is meant that a polypeptide coding sequence and minimal transcriptional and translational controlling sequences are connected in such a way as to permit polypeptide expression when the appropriate molecules (e.g., transcriptional activator proteins) are bound to the regulatory sequence(s). In the present invention, polypeptide expression in a target vertebrate cell is particularly preferred.

The term “isolated DNA” means DNA that is free of the genes and other nucleotide sequences that flank the gene in the naturally occurring genome of the organism from which the isolated DNA of the invention is derived. The term therefore includes, for example, a recombinant DNA which is incorporated into a vector; into an autonomously replicating plasmid or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA that is part of a hybrid gene encoding additional polypeptide sequences.

Accordingly preferred embodiments of the invention, DNA vaccines, consisting of DNA transcription units, are administered to an individual in whom immunization and protection is desired.

A DNA transcription unit is a polynucleotide sequence, bounded by an initiation site and a termination site that is transcribed to produce a primary transcript. As used herein, a “DNA transcription unit” includes at least two components: (1) peptide-encoding DNA, and (2) a transcriptional promoter element or elements operatively linked for expression of the peptide-encoding DNA. Peptide-encoding DNA can encode one or multiple peptides, such as peptides described herein. The DNA transcription unit can additionally be inserted into a vector, which includes sequences for expression of the DNA transcription unit. A DNA transcription unit can optionally include additional sequences such as enhancer elements, splicing signals, termination and polyadenylation signals, viral replicons, and bacterial plasmid sequences. In the present method, a DNA transcription unit (i.e., one type of transcription unit) can be administered individually or in combination with one or more other types of DNA transcription units or in combination with other vaccines, conventional or otherwise. DNA transcription units can be produced by a number of known methods. For example, DNA encoding the desired peptide can be inserted into an expression vector (see, for example. Sambrook et al. Molecular Cloning. A Laboratory Manual. 2d. Cold Spring Harbor Laboratory Press (1989)). With the availability of automated nucleic acid synthesis equipment, DNA can be synthesized directly when the nucleotide sequence is known, or by a combination of polymerase chain reaction (PCR), cloning, and fermentation. Moreover, when the sequence of the desired polypeptide is known, a suitable coding sequence for the polynucleotide can be inferred. Standard molecular biology techniques may be used to replace codons in prokaryotic DNA with eukaryotic codons for the same respective amino acid. Standard molecular biology techniques may be used to engineer protein translation and subsequent host cell modifications such as glycosylation, myristoylation, or phosphorylation to optimize extracellular host cell expression.

The DNA transcription unit can be administered to an individual, or inoculated, in the presence of adjuvants or other substances that have the capability of promoting DNA uptake or recruiting immune system cells to the site of the inoculation. It should be understood that the DNA transcription unit itself is expressed in the host cell by transcription factors provided by the host cell, or provided by a DNA transcription unit.

The “desired peptide” can be any peptide or combination of peptides, which are described herein. The encoded peptides can be translation products or polypeptides. The polypeptides can be of various lengths, and can undergo normal host cell modifications such as glycosylation, myristoylation, or phosphorylation. In addition, they can be designated to undergo intracellular, extracellular, or cell-surface expression. Furthermore, they can be designed to undergo assembly and release from cells.

Immunizations as described herein are accomplished with various DNA transcription units encoded on plasmid vectors that express different peptides described herein. The DNA transcription units described herein are representative of the types of transcription units that can be used in the current invention. The DNA transcription units can encode a single peptide and can additionally encode a combination of peptides.

The methods of preferred embodiments of the invention may be applied by direct injection of the polynucleotide into cells of the animal in vivo, or by in vitro transfection of some of the animal cells which are then re-introduced into the animal body. The DNA may be delivered to various cells of the animal body, including muscle, skin, brain, lung, liver, spleen, or to some cells of the blood. Delivery of the DNA directly in vivo is preferably to the cells of muscle or skin. The polynucleotides may be injected into muscle or skin using an injection syringe. They may also be delivered into muscle or skin using a vaccine gun.

The optimal concentration of the peptide or peptides will necessarily depend upon the specific peptide(s) used, the characteristics of the patient, and the nature or particular SARS-CoV-2 virus. These factors can be determined by those of skill in the medical and pharmaceutical arts in view of the present disclosure. In general, the peptides are most desirably administered at a concentration level that will generally afford antiviral effective results against the selected virus(es) without causing any harmful or deleterious side effects. Specific dosages may be adjusted depending on conditions of disease, the age, body weight, general health conditions, sex, diet of the subject, dose intervals, administration routes, excretion rate, and combinations of drugs. Any of the above dosage forms containing effective amounts are well within the bounds of routine experimentation and therefore, well within the scope of the instant disclosure. A therapeutically effective dose may vary depending upon the route of administration and dosage form.

It will be further appreciated that the amount of an peptide of the present disclosure that is useful will vary not only with the particular peptide selected but also with the route of administration, and the age and condition of the patient, and will ultimately be at the discretion of the attendant physician. In general however, a suitable dose will be in the range of from about 0.01 to 750 mg/kg of body weight per day preferably in the range of 0.1 to 100 mg/kg/day, most preferably in the range of 0.5 to 25 mg/kg/day.

The peptides of the present disclosure may be administered therapeutically or prophylactically. Treatment is preferably commenced before or at the time of infection. However, the treatment can also be commenced post-infection, after the exposure to a virus that is capable of causing a viral respiratory infection, or after the appearance of established symptoms of infection. Suitable treatment is given 1-4 times daily and continued for 1-10 days, and typically 8 days post infection. Suitable prophylactic administration is given in single or multiple doses that may be spaced apart in accordance with known booster vaccination schedules and/or given annually in accordance with vaccination schedules. The desired dose may be presented in a single dose or as divided doses administered at appropriate intervals, for example as two, three, four or more sub-doses per day.

The peptide may be conveniently administered in unit dosage form, for example, containing 10 to 1500 mg, conveniently 20 to 1000 mg, most conveniently 50 to 700 mg of active ingredient per unit dosage form, e.g. 1 mg/kg equates to 75 mg/75 kg of body weight.

In one aspect, a method of eliciting an immune response is provided by administering an immunologic composition comprising a peptide having an informational spectrum (IS) that overlaps with the IS of SP1 protein or fragment thereof, wherein the peptide is capable of eliciting an immune response against the SP1 protein or fragment thereof. The SP1 protein and the peptide may be homologous such that the peptide is suitable for use as an antigen to produce antibodies, preferably neutralizing antibodies that bind to the SP1 protein or fragment thereof.

In one aspect, a cross-spectrum (CS) based on the IS of the SP1 protein or fragment thereof (determined using an ISM approach as described herein) and the IS of the peptide may contain frequency components of F(0.257) and F(0.479). Additionally, the IS of the peptide may comprise F(0.255-0.259) and F(0.477-0.481).

In one aspect, a peptide may comprise 36 to 646 amino acids. More particularly, the peptide may comprise 15 to 65 amino acids.

In one aspect, the peptide has the sequence of SEQ ID NO: 1, which is:
LKYNENGTITDAVDCALDPLSETKCTLKSFTVEKGIYQTSNFRVQPTESIVRFPN
ITNLCPFGEVFNATRFASVY.
In one aspect, the peptide has the sequence of SEQ ID NO: 2, which is:
DITPCSFGGVSVITPGTNTSNQVAVLYQGVNCTEVPVAIHADQLTPTWRVYSTG
SNVFQTRAGCL.
In one aspect, the peptide has the sequence of SEQ ID NO: 3, which is:
FLLKYNENGTITDAVDCALDPLSETKCTLKSFTVEKGIYQTSNFRVQPTESIVRF
PNITNLCPFGEVFNATRFASVYAWNRKRISNCVA.
In one aspect, the peptide has the sequence of SEQ ID NO: 4, which is:
DHWADKYWRAAMSWHRWWPDYQPDYWHDYYYRWHQMQERQMNRWQER
WWHRQAYSSPQQTESWRIDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 5, which is:
DHWADKYWRAAMSWHRWWPDYQPDYWHDYYYRWHQMQERQMNRWQER
WWHRQAYSSPQLTPTWRVDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 6, which is:
DHWADKYWRAAMSWHRWWPDYQPDYWHDYYYRWHQMQERQMNRWQER
WWHRQAGSTPQLTPTWRVDKTHTCPPCPAPELLGGPSVFLFPPKPKDTLMISR
TPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 7, which is:
DHWADKYWRAAMSWHRWWPDYQPDYWHDYYYRWHQMQERQMNRWQER
WWHRQAYSSPQQTESWRI.
In one aspect, the peptide has the sequence of SEQ ID NO: 8, which is:
SWHRWWPDYQPDYWHDYYYRWHQMQERQMNRWQERWWHRQAYSSPQLT
PTWRV.
In one aspect, the peptide has the sequence of SEQ ID NO: 9, which is:
DHWADKYWRAAMSWHRWWPDYQPDYWHDYYYRWHQMQERQMNRWQER
WWHRQAGSTPQLTPTWRV.
In one aspect, the peptide has the sequence of SEQ ID NO: 10, which is:
FLLKYNENGTITDAVDCALDPLSETKCTLKSFTVEKGIYQTSNFRVQPTESIVRF
PNITNLCPFGSNNLDSKVGGNYNYLYRLFRKSNLKPFERDISTEIYQAGSTPCNG
VEGFNCYFPLQSYGFQPTNGVGYQPY.
In one aspect, the peptide has the sequence of SEQ ID NO: 11, which is:
FLLKYNENGTITDAVDCALDPLSETKCTLKSFTVEKGIYQTSNFRVQPTESIVRF
PNITNLCPFGEVFNATRFASVYAWNRKRISNCVASNNLDSKVGGNYNYLYRLF
RKSNLKPFERDISTEIYQAGSTPCNGVEGFNCYFPLQSYGFQPTNGVGYQPY.
In one aspect, the peptide has the sequence of SEQ ID NO: 12, which is:
DHWADKYWRAAMSWHRWWPDYQPDYWHDYYYRWHQMQERQMNRWQER
WWHRQAYSSPQLTPTWRVSNNLDSKVGGNYNYLYRLFRKSNLKPFERDISTEI
YQAGSTPCNGVEGFNCYFPLQSYGFQPTNGVGYQPY.
In one aspect, the peptide has the sequence of SEQ ID NO: 13, which is:
DHWADKYWRAAMSWHRWWPDYQPDYWHDYYYRWHQMQERQMNRWQER
WWHRQAGSTPQLTPTWRVSNNLDSKVGGNYNYLYRLFRKSNLKPFERDISTEI
YQAGSTPCNGVEGENCYFPLQSYGFQPTNGVGYQPY.
In one aspect, the peptide has the sequence of SEQ ID NO: 14, which is:
DHWADKYWRAAMSWHRWWPDYQPDYWHDYYYRWHQMQERQMNRWQER
WWHRQAYSSPQQTESWRIRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 15, which is:
DHWADKYWRAAMSWHRWWPDYQPDYWHDYYYRWHQMQERQMNRWQER
WWHRQAYSSPQLTPTWRVRVQPTESIVRFPNITNLCPFGEVENATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 16, which is:
DHWADKYWRAAMSWHRWWPDYQPDYWHDYYYRWHQMQERQMNRWQER
WWHRQAGSTPQLTPTWRVRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 17, which is:
DKQADKWWDYATTWHDQQPDWQPDYQKDWYWRQHSTMERQTGDWSEDQ
QHDQYYRTHQMRERQRI.
In one aspect, the peptide has the sequence of SEQ ID NO: 18, which is:
DKQADKWWDYATTWHDQQPDWQPDYQKDWYWRQHSTMERQTGDWSEDQ
QHDQYYRTHQMRERQRISNNLDSKVGGNYNYLYRLFRKSNLKPFERDISTEIY
QAGSTPCNGVEGENCYFPLQSYGFQPTNGVGYQPY.
In one aspect, the peptide has the sequence of SEQ ID NO: 19, which is:
DKQADKWWDYATTWHDQQPDWQPDYQKDWYWRQHSTMERQTGDWSEDQ
QHDQYYRTHQMRERQRIRVQPTESIVRFPNITNLCPFGEVENATRFASVYAWN
RKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLS
FELLHAPATVCGPKKSTNLVKNK.
In one aspect, the peptide has the sequence of SEQ ID NO: 20, which is:
DKQADKWWDYATTWHDQQPDWQPDYQKDWYWRQHSTMERQTGDWSEDQ
QHDQYYRTHQMRERQRISNNLDSKVGGNYNYLYRLFRKSNLKPFERDISTEIY
QAGSTPCNGVEGENCYFPLQSYGFQPTNGVGYQPYDKTHTCPPCPAPELLGGP
SVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
In one aspect, the peptide has the sequence of SEQ ID NO: 21, which is:
SQCVNDTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQ
PFLMDLEGKQGNFKNLREFVFKNIDGYFKIYSKHTPINLVRDLPQGFSALEPLV
DLPIGINITRFQTLLADHRSYLTPGDSSSGWTAGAAAYYVGYLQPRTFLLKYNE
NGTITDAVDCALDPLSETKCTLKSFTVEKGIYQTSNFRVQPTESIVRFPNITNLCP
FGEVFNATRFASV.
In one aspect, the peptide has the sequence of SEQ ID NO: 22, which is:
YAWNRKRISNCVADYSVLYNSASFSTFKCYGVSPTKLNDLCFTNVY AISFVIRG
DEVRQIAPGQTGKIADYNYKLPDDFTGCVIAWNSNNLDSKVGGNYNYLYRLF
RKSNLKPFERDISTEIYQAGSTPCNGVEGENCYFPLQSYGFQPTNGVGYQPYRV
VVLSFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQGVNC
TEVPVAIHADQLTPTWRVYSTGSNVFQTRAGCLIGAEHVNNSYECDIPIGAGIC
ASYQTQTNSPRRAR.
In one aspect, the peptide has the sequence of SEQ ID NO: 23, which is:
GKQGNFKNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTITDAV
DCALDPLSETKCTLKSFTVEKGIYQTSNFRVQPTESIVRFPNITNL.
In one aspect, the peptide has the sequence of SEQ ID NO: 24, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TLAVDCALDPLSETKCTLKSFTVEKGIYQTSNFRVQPTEVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYF.
In one aspect, the peptide has the sequence of SEQ ID NO: 25, which is:
PLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVI
TPGTNTSNQVAVLYQDVNCTEVPVAIHADQLTPTWRVYSTGSNVFQTRAGCLI
GAEHVNNSYE.
In one aspect, the peptide has the sequence of SEQ ID NO: 26, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TLAVDCALDPLSETKCTLKSFTVEKGIYQTSNFRVQPTIVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVLTES
NKKFLPFQQFGRDIADTTDAVRDPQTLEILDITPCSFGGVSVITPGTNTSNQVAV
LYQDVNCTEVPVAIHADQLTPTWRVYSTGSNVFQTRAGCLIGAEHVNNSYE.
In one aspect, the peptide has the sequence of SEQ ID NO: 27, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TDAVDCALDPLSETKCTLKSFTVEKGIYQTSNFRVQPTIVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVLTES
NKKFLPFQQFGRDIADTTDAVRDPQTLEILDITPCSFGGVSVITPGTNTSNQVAV
LYQDVNCTEVPVAIHADQLTPTWRVYSTGSNVFQTRAGCLIGAEHVNNSYE.
In one aspect, the peptide has the sequence of SEQ ID NO: 28, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TDAVDCALDPLSETKCTLKSFTVEKGIYQTSNFRVQPTEVRFPNITNLCPFGEVF
NATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVLTES
NKKFLPFQQFGRDIADTTDAVRDPQTLEILDITPCSFGGVSVITPGTNTSNQVAV
LYQDVNCTEVPVAIHADQLTPTWRVYSTGSNVFQTRAGCLIGAEHVNNSYE.
In one aspect, the peptide has the sequence of SEQ ID NO: 29, which is:
VNLTTRTQDPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 30, which is:
VNLTTRTQLPPAYTNSFTRGVYYPDKVFRSSVLHSTQLLFLPFFSNVTWFHAIH
VSGTNGTKRFDNPVLPFNLGVYFASTEKSNIIRGWIFGTTLDSKTQSLLIVNNAT
NVVIKVCEFQFCNDPFLGVYYHKNNKSMESEFRVYSSANNCTFEYVSQPFLMD
LEGKQGNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENGTIT
DAVDCALDPLSETKCTLKSFTVEKGIYQTSNFRVQPTESIVRFPNITNLCPFGEV
FNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQV
AVLYQDVNCTEVPVAIHADQLTPTWRVYSTGSNVFQTRAGCLIGAEHVNNSY
E.
In one aspect, the peptide has the sequence of SEQ ID NO: 31, which is:
VNLTTRTQDPPAYTNSFTRGVYYPDKVFRSSVLHSTQDLFLPFFSNVTWFHAIH
VSGTNGTKRFDNPVLPFNLGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 32, which is:
VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 33, which is:
VNLTLRTQLPPAYTNSFTRGVYYPDKVFRSSVLHSTQLLFLPFFSNVTWFHAIH
VSGTNGTKRFDNPVLPFNLGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLDSKVG
GNYNYLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 34, which is:
VNLTTRTQDPPAYTNSFTRGVYYPDKVFRSSVLHSTQDLFLPFFSNVTWFHAIH
VSGTNGTKRFDNPVLPFNLGVYFASTEKSNIIRGWIFGTTLDSKTQSDLDVNNA
TNVVIKVCEFQFCNDPFLGVYYHKNNKSWMESEFRVYSSANNCTFEYVSQPFL
MDLEGKQGNFKNLREFVFKNIDGYFKDYSKHTPINLVRDLPQGFSALEPLVDL
PIGINITRFQTLDALHRSYLTPGDSSSGWTAGAAAYYVGYLQPRTFLDKYNENG
TITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNS
YE.
In one aspect, the peptide has the sequence of SEQ ID NO: 35, which is:
LKYNENGTITDAVDCALDPLSETKCTLKSFTVEKGIYQTSNFRVQPTESIVRFPN
ITNLCPFGEVENATRFASVY.
In one aspect, the peptide has the sequence of SEQ ID NO: 36, which is:
DITPCSFGGVSVITPGTNTSNQVAVLYQGVNCTEVPVAIHADQLTPTWRVYSTG
SNVFQTRAGCL.
In one aspect, the peptide has the sequence of SEQ ID NO: 37, which is:
FLLKYNENGTITDAVDCALDPLSETKCTLKSFTVEKGIYQTSNFRVQPTESIVRF
PNITNLCPFGEVFNATRFASVYAWNRKRISNCVA.
In one aspect, the peptide has the sequence of SEQ ID NO: 38, which is:
DHWADKYWRAAMSWHRWWPDYQPDYWHDYYYRWHQMQERQMNRWQER
WWHRQAYSSPQQTESWRIDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 39, which is:
DHWADKYWRAAMSWHRWWPDYQPDYWHDYYYRWHQMQERQMNRWQER
WWHRQAYSSPQLTPTWRVDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 40, which is:
DHWADKYWRAAMSWHRWWPDYQPDYWHDYYYRWHQMQERQMNRWQER
WWHRQAGSTPQLTPTWRVDKTHTCPPCPAPELLGGPSVFLFPPKPKDTLMISR
TPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVD
KSRWQQGNVFSCSVMHEALHNHYTQKSLSLSPGK.
In one aspect, the peptide has the sequence of SEQ ID NO: 41, which is:
DHWADKYWRAAMSWHRWWPDYQPDYWHDYYYRWHQMQERQMNRWQER
WWHRQAYSSPQQTESWRI.
In one aspect, the peptide has the sequence of SEQ ID NO: 42, which is:
SWHRWWPDYQPDYWHDYYYRWHQMQERQMNRWQERWWHRQAYSSPQLT
PTWRV.
In one aspect, the peptide has the sequence of SEQ ID NO: 43, which is:
DHWADKYWRAAMSWHRWWPDYQPDYWHDYYYRWHQMQERQMNRWQER
WWHRQAGSTPQLTPTWRV.
In one aspect, the peptide has the sequence of SEQ ID NO: 44, which is:
FLLKYNENGTITDAVDCALDPLSETKCTLKSFTVEKGIYQTSNFRVQPTESIVRF
PNITNLCPFGSNNLDSKVGGNYNYLYRLFRKSNLKPFERDISTEIYQAGSTPCNG
VEGFNCYFPLQSYGFQPTNGVGYQPY.
In one aspect, the peptide has the sequence of SEQ ID NO: 45, which is:
FLLKYNENGTITDAVDCALDPLSETKCTLKSFTVEKGIYQTSNFRVQPTESIVRF
PNITNLCPFGEVFNATRFASVYAWNRKRISNCVASNNLDSKVGGNYNYLYRLF
RKSNLKPFERDISTEIYQAGSTPCNGVEGENCYFPLQSYGFQPTNGVGYQPY.
In one aspect, the peptide has the sequence of SEQ ID NO: 46, which is:
DHWADKYWRAAMSWHRWWPDYQPDYWHDYYYRWHQMQERQMNRWQER
WWHRQAYSSPQLTPTWRVSNNLDSKVGGNYNYLYRLFRKSNLKPFERDISTEI
YQAGSTPCNGVEGENCYFPLQSYGFQPTNGVGYQPY.
In one aspect, the peptide has the sequence of SEQ ID NO: 47, which is:
DHWADKYWRAAMSWHRWWPDYQPDYWHDYYYRWHQMQERQMNRWQER
WWHRQAGSTPQLTPTWRVSNNLDSKVGGNYNYLYRLFRKSNLKPFERDISTEI
YQAGSTPCNGVEGFNCYFPLQSYGFQPTNGVGYQPY.
In one aspect, the peptide has the sequence of SEQ ID NO: 48, which is:
DHWADKYWRAAMSWHRWWPDYQPDYWHDYYYRWHQMQERQMNRWQER
WWHRQAYSSPQQTESWRIRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 49, which is:
DHWADKYWRAAMSWHRWWPDYQPDYWHDYYYRWHQMQERQMNRWQER
WWHRQAYSSPQLTPTWRVRVQPTESIVRFPNITNLCPFGEVFNATRFASVYAW
NRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRVVVL
SFELLHAPATVCGPKKSTNLVKNK.
In one aspect, the peptide has the sequence of SEQ ID NO: 50, which is:
DHWADKYWRAAMSWHRWWPDYQPDYWHDYYYRWHQMQERQMNRWQER
WWHRQAGSTPQLTPTWRVRVQPTESIVRFPNITNLCPFGEVFNATRFASVYAW
NRKRISNCVADYSVLYNSASFSTFKCYGVSPTKLNDLCFTNVY ADSFVIRGDEV
RQIAPGQTGKIADYNYKLPDDFTGCVIAWNSNNLDSKVGGNYNYLYRLFRKS
NLKPFERDISTEIYQAGSTPCNGVEGENCYFPLQSYGFQPTNGVGYQPYRVVVL
SFELLHAPATVCGPKKSTNLVKNK.
In one aspect, the peptide has the sequence of SEQ ID NO: 51, which is:
DKQADKWWDYATTWHDQQPDWQPDYQKDWYWRQHSTMERQTGDWSEDQ
QHDQYYRTHQMRERQRI.
In one aspect, the peptide has the sequence of SEQ ID NO: 52, which is:
DKQADKWWDYATTWHDQQPDWQPDYQKDWYWRQHSTMERQTGDWSEDQ
QHDQYYRTHQMRERQRISNNLDSKVGGNYNYLYRLFRKSNLKPFERDISTEIY
QAGSTPCNGVEGFNCYFPLQSYGFQPTNGVGYQPY.
In one aspect, the peptide has the sequence of SEQ ID NO: 53, which is:
DKQADKWWDYATTWHDQQPDWQPDYQKDWYWRQHSTMERQTGDWSEDQ
QHDQYYRTHQMRERQRIRVQPTESIVRFPNITNLCPFGEVFNATRFASVYAWN
RKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLS
FELLHAPATVCGPKKSTNLVKNK.
In one aspect, the peptide has the sequence of SEQ ID NO: 54, which is:
DKQADKWWDYATTWHDQQPDWQPDYQKDWYWRQHSTMER
QTGDWSEDQQHDQYYRTHQMRERQRISNNLDSKVGGNYNYLYRLFRKSNLK
PFERDISTEIYQAGSTPCNGVEGENCYFPLQSYGFQPTNGVGYQPYDKTHTCPP
CPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQK
SLSLSPGK.
In one aspect, the peptide has the sequence of SEQ ID NO: 55, which is:
SQCVNDTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQ
PFLMDLEGKQGNFKNLREFVFKNIDGYFKIYSKHTPINLVRDLPQGFSALEPLV
DLPIGINITRFQTLLADHRSYLTPGDSSSGWTAGAAAYYVGYLQPRTFLLKYNE
NGTITDAVDCALDPLSETKCTLKSFTVEKGIYQTSNFRVQPTESIVRFPNITNLCP
FGEVFNATRFASVYAWNRKRISNCVADYSVLYNSASFSTFKCYGVSPTKLNDL
CFTNVY AISFVIRGDEVRQIAPGQTGKIADYNYKLPDDFTGCVIAWNSNNLDSK
VGGNYNYLYRLFRKSNLKPFERDISTEIYQAGSTPCNGVEGENCYFPLQSYGFQ
PTNGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITPGTNTSN
QVAVLYQGVNCTEVPVAIHADQLTPTWRVYSTGSNVFQTRAGCLIGAEHVNN
SYECDIPIGAGICASYQTQTNSPRRAR.
In one aspect, the peptide has the sequence of SEQ ID NO: 56, which is:
GKQGNFKNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTITDAV
DCALDPLSETKCTLKSFTVEKGIYQTSNFRVQPTESIVRFPNITNL.
In one aspect, the peptide has the sequence of SEQ ID NO: 57, which is:
GKQGNFKNLREFVFKNIDGYFKIYSKHTPINLDRILPQGFSALEPLVDLPIGINIT
RFQTLLADHRSYLTPGDSSSGWTAGAAAYYDGYLQPRTFLLKYNENGTITDAV
DCALDPLSETKCTLKSFTVEKGIYQTSNFRVQPTESIVRFPNITNL.

In one aspect, the peptide of the immunologic composition is suitable for use as an antigen in a vaccine. The peptide may be used alone or in combination with one or more other SARS-CoV-2 antigens. The resulting immunologic composition may provide protection against one or more virus strains.

In another aspect, the immunologic composition may further comprise a pharmaceutically acceptable carrier and/or excipient and/or adjuvant. Additionally, the immunologic composition may further comprise another antigen obtained from SARS-Cov-2 (e.g., ORF3b, ORF6, ORF8).

Further aspects provide for a method for producing the immunologic composition comprising (i) obtaining the peptide, and (ii) admixing the peptide with a pharmaceutically acceptable carrier and/or excipient and/or adjuvant.

Additional aspects contemplate a peptide that (i) comprises an amino acid sequence whose informational spectrum (IS) contains a frequency components F(0.257) and F(0.479), and (ii) produces antibodies that bind to a SP1 protein of SARS-CoV-2. The antibodies produced are preferably neutralizing antibodies. The peptide may be suitable for use as an antigen in an immunologic composition.

Certain aspects further encompass a method of using at least one peptide as an immunogen in order to generate antibodies. e.g., neutralizing antibodies that specifically bind to SP1 protein.

Additionally, certain aspects contemplate an isolated nucleic acid encoding the peptide: a vector containing the nucleic acid; and an isolated cell containing the vector as well as an antibody or antigen binding fragment thereof capable of binding to the peptide.

One aspect contemplates a therapeutic method or diagnostic method using the peptide or the antibody or antigen binding fragment thereof that binds to the peptide. For example, the peptide or the antibody or antigen binding fragment thereof that binds to the peptide are suitable for use a variety of diagnostic methods, including but not limited to, immunoassay's such as ELISA, ELISPOT, enzyme multiplied immunoassay technique, radioimmunoassay, and immunofluorescence; immunoblotting assays (such as a Western blot); immunoprecipitation assays such as chromatin immunoprecipitation (ChIP), immunodiffusion, and immunoelectrophoresis; immunocytochemistry; and immunohistochemistry. These diagnostic methods (and other similar methods) involve applying the peptide or the antibody or antigen binding fragment thereof that binds to the peptide to a sample (e.g., serum or mucosal) collected from a patient believed to have SARS-CoV-2 in order to detect and characterize the infection.

Another aspect encompasses a method for identifying a peptide that elicits an immune response against SP1 protein or fragment thereof. The method comprises (1) (i) obtaining an amino acid sequence of SP1 protein or fragment thereof; (ii) assigning an electron-ion interaction potential (EIIP) index value to each amino acid residue contained in the amino acid sequence of the SP1 protein or fragment; (iii) subjecting the resultant EIIP index values to discrete Fourier transformation (DFT); (iv) generating an informational spectrum (IS) of the SP1 protein or fragment based on the EIIP index values; (2) (i) obtaining an amino acid sequence of a peptide or peptides; (ii) assigning an EIIP index value to each amino acid residue contained in the amino acid sequence of the peptide or peptides; (iii) subjecting the resultant EIIP index values to discrete Fourier transformation (DFT); (iv) generating an informational spectrum (IS) of each peptide based on the EIIP index values: (3) comparing the IS of the SP1 protein or fragment generated in (1) (iv) to the IS of the peptide or peptides generated in (2) (iv); and (4) identifying the peptide or peptides whose IS overlap with the IS of the SP1 protein or fragment, and based thereon identifying the peptide or peptides as one being capable of eliciting an immune response against the SP1 protein or fragment.

The following examples are offered to illustrate, but not to limit, the claimed invention.

### EXAMPLES

### Example 1: Analysis of Amino Acid Sequences Using the Informational Spectrum Method (ISM)

ISM is based on the premise that the protein-protein interaction encompasses two basic steps: (i) recognition and targeting between interacting proteins (long-range interactions at distances >100 Å) and (ii) chemical binding (short range interactions at distances <5 Å). The long-range properties of biological molecules are determined by the electron-ion interaction potential (EIIP) representing the main energy term of valence electrons. The EIIP for organic molecules can be calculated by the following simple equation derived from the “general model pseudopotential”:

$$W = 0.25 {Z}^{*} sin ( 1.04 π {Z}^{*} ) / 2 π \qquad ( 1 )$$

where Z* is the average quasivalence number (AQVN) determined by

$${Z}^{*} {\sum }^{m} {n}_{i} {Z}_{i} / N \qquad ( 2 )$$

where Zi is the valence number of the i-th atomic component, ni is the number of atoms of the i-th component, in is the number of atomic components in the molecule, and N is the total number of atoms.

Applying the given expressions (1) and (2) to 20 amino acids, the following EIIP values are obtained (in Ry): L 0.0000, 10.0000, N 0.0036, G 0.0050, V 0.0057, E 0.0058. P 0.0198, H 0.0242, K 0.0371, A 0.0373, Y 0.0516, W 0.0548, Q 0.0761, M 0.0823. S 0.0829, C 0.0829, T 0.0941, F 0.0946, R 0.0959 and D 0.1263. The ISM technique is based on a model of the primary structure of a protein using a sequence of numbers, by assigning to each amino acid the correspondence value of EIIP. The obtained numerical sequence, representing primary structure of protein, is then subjected to a discrete Fourier transformation which is defined as follows:

$$X ( n ) = \sum x ( m ) {e}^{- j ( 2 / N ) nm} , n is 1 , 2 , … , N / 2 \qquad ( 3 )$$

where x(m) is the m-th member of a given numerical series, N is the total number of points in this series, and X(n) are discrete Fourier transformation coefficients.

These coefficients describe the amplitude, phase and frequency of sinusoids, which comprised the original signal. The absolute value of a complex discrete Fourier transformation defines the amplitude spectrum and the phase spectrum. The complete information about the original sequence is contained in both spectral functions. However, in the case of protein analysis, relevant information is presented in an energy density spectrum, which is defined as follows:

$$S ( n ) = X ( n ) X * ( n ) is {\vert X ( n ) \vert}^{2} , n is 1 , 2 , … , N / 2. \qquad ( 4 )$$

In this way, sequences are analyzed as discrete signals. It is assumed that their points are equidistant with the distance d is 1. The maximal frequency in a spectrum defined as above is F is ½d is 0.5. The frequency range is independent of the total number of points in the sequence. The total number of points in a sequence influences only the resolution of the spectrum. The resolution of the N-point sequence is 1/n. The n-th point in the spectral function corresponds to a frequency f (n) is of is n/N. Thus, the initial information defined by the sequence of amino acids can now be presented in the form of the informational spectrum (IS), representing the series of frequencies and their amplitudes.

The IS frequencies correspond to the distribution of structural motifs with defined physicochemical properties determining a biological function of a protein. When comparing proteins, which share the same biological or biochemical function, the ISM technique allows detection of code/frequency pairs which are specific for their common biological properties, or which correlate with their specific interaction. These common informational characteristics of sequences are determined by cross-spectrum or consensus informational spectrum (CIS). A CIS of N spectra is obtained by the following equation:

$$C ( j ) = \prod S ( i , j ) , \qquad ( 5 )$$

where Π(i,j) is the j-th element of the i-th power spectrum and C(j) is the j-th element of CIS.

Thus, CIS is the Fourier transform of the correlation function for the spectrum. Thus, any spectral component (frequency) not present in all compared informational spectra is eliminated. Peak frequencies in CIS represent the common information encoded in the primary structure of analyzed sequences. This information corresponds to the mutual long-range interaction between analyzed proteins or their interaction with the common interactor.

ISM analysis provides the ability to: (i) predict the biological function of protein: (ii) compare the biological activity within a group of proteins with the same function: (iii) predict mutations which could increase or decrease biological activity of protein; and (iv) design an artificial protein sequence with desired biological function.

### Example 2: Use of ISM to Analyze SP1 from SARS-CoV-2 Viruses

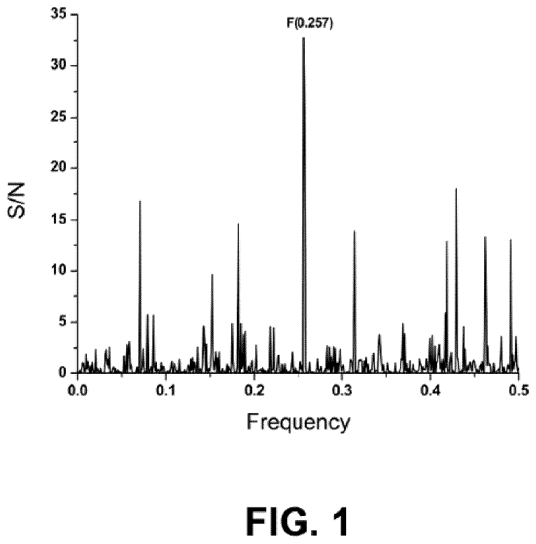

The analysis comprised the following steps: 1. each amino acid sequence was converted to the numerical sequence by representing each amino acid with the corresponding value of the EIIP: 2. this numerical sequence was converted into a numerical spectrum using fast Fourier transform (FFT); and 3. spectra were mutually compared using cross-spectral analysis with the aim to extract common frequency components. From the ISM analysis of SP1 protein from SARS-Cov-2, SARS-CoV-1 and MERS virus and ACE2 receptor (see FIG. 1), the frequency F(0.257) was identified as the common frequency component which determines virus/receptor interaction.

### Example 3: Comparison of SP12 Protein from SARS-CoV-1 and SARS-CoV-2 by ISM

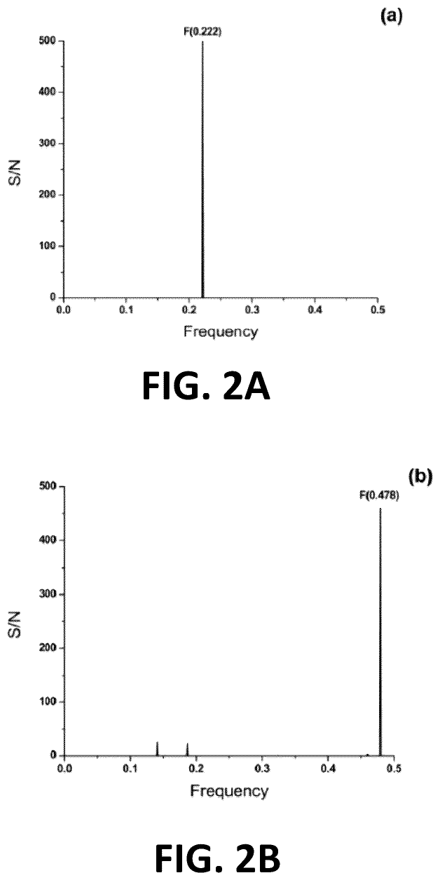

CS of SI spike proteins from SARS-CoV-1 and SARS-CoV-2 were compared (FIGS. 2a and 2b). Principal information encoded in SI proteins from these viruses is represented with two different frequencies F(0.222) and F(0.479), respectively. This result indicates some potential difference(s) in the virus-host interaction of these two viruses although they apparently use the same receptor ACE2.

### Example 4: SARS-CoV-2 Challenge in Hamsters

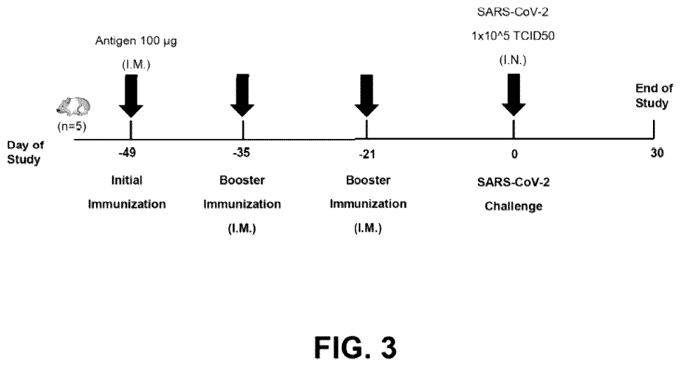

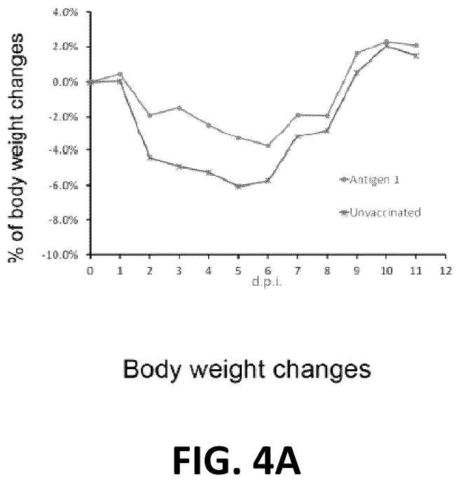

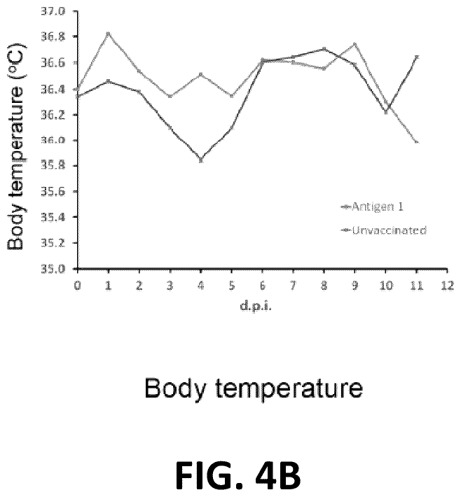

The embodiments relating to the methods of treatment described herein were demonstrated in Syrian golden hamsters. Syrian golden hamsters are an excellent small animal model to study SARS-CoV-2 infection due to their low mortality, marked pathology, and competent immune response. Hamsters were inoculated with an initial dose 100 μg of antigen followed by two additional boosters as outline in FIG. 3. Following the immunization sequence. all hamsters were inoculated intranasally with 10⁵ 50% tissue culture infectious dose (TCID₅₀) of SARS-CoV-2 (FIG. 3). The changes in % body weight and body temperature were measured in relation to days post-infection with SARS-CoV-2. As can be seen in FIGS. 4(a)-(b) the hamsters which received the antigen immunizations better maintained their weight and body temperature.

The examples given above are merely illustrative and are not meant to be an exhaustive list of all possible embodiments, applications, or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, the various modifications of the described modes of carrying out the invention, which are apparent to those skilled in the relevant fields, are intended to be within the scope of the appended claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. In addition, the materials, methods, and examples are illustrative only and not intended to be limiting.

Since many embodiments of the invention can be made without departing from the spirit and scope of the invention, the invention resides in the claims hereinafter appended.

1. A method of eliciting an immune response by administering an immunologic composition comprising:
a peptide having an informational spectrum (IS) that overlaps with the IS of spike protein subunit 1 (SP1) of SARS-CoV-2 or fragment thereof,
wherein the peptide is capable of eliciting an immune response against the SP1 protein or fragment thereof.

2. The method of claim 1, wherein:
(i) the at least SP1 protein and the peptide are homologous such that the peptide is suitable for use as an antigen to produce antibodies that bind to the SP1 protein or fragment thereof;
(ii) the antibodies produced against any of the foregoing peptides are neutralizing antibodies;
(iii) the SP1 protein or fragment thereof according to any of the foregoing methods is derived from reference SARS-CoV-2 strain Wuhan-HU-1 (YP_009724390);
(iv) a cross-spectrum (CS) based on the informational spectrum (IS) of the SP1 protein or fragment thereof and the IS of the peptide according to any of the foregoing methods contains a frequency components of F(0.257) and F(0.479);
(v) the IS of the peptide according to any of the foregoing methods comprises F(0.255-0.259) and F(0.477-0.481);
(vi) the peptide according to any of the foregoing methods comprises 36 to 646 amino acids; and
(vii) the peptide according to any of the foregoing methods comprises 15 to 50 amino acids.

3. The method of claim 1, wherein the peptide comprises an amino acid sequence selected from SEQ ID NOS: 1 to 23.

4. An immunologic composition comprising:
a peptide having an informational spectrum (IS) that overlaps with the IS of SP1 protein or fragment thereof,
wherein the peptide is capable of eliciting an immune response against the SP1 protein or fragment thereof.

5. The immunologic composition of claim 4, wherein:
(i) the SP1 protein and the peptide are homologous such that the peptide is suitable for use as an antigen to produce antibodies that bind to the SP1 protein or fragment thereof;
(ii) antibodies produced against a peptide contained in the immunologic composition are neutralizing antibodies;
(iii) a cross-spectrum (CS) based on the IS of the SP1 protein or fragment thereof according to any of the foregoing and the IS of the peptide according to any of the foregoing contains a frequency components of F(0.257) and F(0.479);
(iv) the IS of a peptide according to any of the foregoing comprises F(0.255-0.259) and F(0.477-0.481);
(v) a peptide according to any of the foregoing comprises 36 to 646 amino acids; and
(vi) a peptide according to any of the foregoing comprises 15 to 50 amino acids.

6. The immunologic composition according to of claim 4, wherein the peptide comprises an amino acid sequence selected from SEQ ID NOS: 24 to 34.

7. The immunologic composition claim 4, wherein:
(i) the peptide is suitable for use as an antigen in a vaccine;
(ii) the peptide according to any of the foregoing alone or in combination with one or more SARS-CoV-2 vaccine antigens;
(iii) the immunologic composition according to any of the foregoing provides protection against SARS-CoV-2;
(iv) the immunologic composition according to any of the foregoing further comprises a pharmaceutically acceptable carrier and/or excipient and/or adjuvant;
(v) the immunologic composition according to any of the foregoing further comprises another antigen obtained from SARS-CoV-2 virus; or
(vi) the immunologic composition comprises any combination of the foregoing.

8. A method for producing the immunologic composition of claim 4, comprising (i) obtaining the peptide, and (ii) admixing the peptide with a pharmaceutically acceptable carrier or excipient or adjuvant.

9. A peptide that:
(i) comprises an amino acid sequence whose informational spectrum (IS) contains a frequency components F(0.357) and F(0.479), and
(ii) produces antibodies that bind to a SP1 protein of SARS-CoV-2 virus.

10. The peptide of claim 9, wherein:
(i) antibodies produced against the peptide include neutralizing antibodies;
(ii) the peptide of claim 9, which is suitable for use as an antigen in an immunologic composition;
(iii) the peptide of claim 9, which comprises 35 to 646 amino acids;
(iv) the peptide of claim 9, which comprises 15 to 50 amino acids; and
(v) the peptide of claim 9, wherein the IS comprises the frequency components selected from F(0.255-0.259) and F(0.477-0.481).

11. A method of using the peptide of claim 9 as an immunogen in order to generate antibodies, optionally neutralizing antibodies, which specifically bind to SP1 protein.

12. An isolated nucleic acid encoding the peptide of claim 9.

13. A vector containing a nucleic acid according to claim 12.

14. An isolated cell containing a vector according to claim 13.

15. An antibody or antigen binding fragment thereof capable of binding to the peptide of claim 9.

16. A therapeutic or diagnostic method which comprises the use of a therapeutically or diagnostically effective amount of the peptide of claim 9.

17. A method for identifying a peptide that elicits an immune response against SP1 protein or fragment thereof, comprising the following:
(1) (i) obtaining an amino acid sequence of SP1 protein or fragment thereof; (ii) assigning an electron-ion interaction potential (EIIP) index value to each amino acid residue contained in the amino acid sequence of the SP1 protein or fragment; (iii) subjecting the resultant EIIP index values to discrete Fourier transformation (DFT); (iv) generating an informational spectrum (IS) of the SP1 protein or fragment based on the EIIP index values;
(2) (i) obtaining an amino acid sequence of a peptide or peptides; (ii) assigning an EIIP index value to each amino acid residue contained in the amino acid sequence of the peptide or peptides; (iii) subjecting the resultant EIIP index values to discrete Fourier transformation (DFT); (iv) generating an informational spectrum (IS) of each peptide based on the EIIP index values;
(3) comparing the IS of the SP1 protein or fragment generated in (1) (iv) to the IS of the peptide or peptides generated in (2) (iv); and
(4) identifying the peptide or peptides whose IS overlap with the IS of the SP1 protein or fragment, and based thereon identifying the peptide or peptides as one being capable of eliciting an immune response against the SP1 protein or fragment.

18. The method of claim 17, which further includes any of the following:
(i) synthesizing at least one of the identified peptides;
(ii) synthesizing at least one of the identified peptides and assessing its immunogenicity or ability to generate antibodies that specifically bind to said SP1 protein;
(iii) the antibodies produced against any of the peptides identified by any of the foregoing methods are neutralizing antibodies;
(iv) producing an immunologic composition comprising at least one of the peptides identified by any of the foregoing methods;
(v) the SP1 protein or fragment identified according to any of the foregoing methods is derived from SARS-CoV-2 virus;
(vi) generating a cross-spectrum (CS) based on the IS of the SP1 protein generated in (1) (iv) and the IS of the peptide or peptides generated in (2) (iv) according to any of the foregoing methods to identify a common frequency component;
(vii) the SP1 protein and the peptide or peptides identified according to any of the foregoing methods are homologous such that the peptide or peptides are suitable for use as an antigen to produce antibodies that bind to the SP1 protein;
(viii) the antibodies produced against peptides identified by any of the foregoing methods are neutralizing antibodies;
(ix) the common frequency components of the peptides identified by any of the foregoing methods is F(0.257) and F(0.479);
(x) the IS of the peptides comprises a series of frequency components (F) that characterize the structural motifs within the SP1 protein and the peptide or peptides;
(xi) the peptide identified according to any of the foregoing methods comprises 35 to 646 amino acids;
(xii) the peptide identified according to any of the foregoing methods comprises 15 to 65 amino acids; and
(xiii) the IS of a peptide identified according to any of the foregoing methods comprises F(0.255-0.259) and F(0.477-0.481).

19-21. (canceled)

22. A method for the treatment or prevention of SARS-CoV-2 virus infection in a subject, comprising:
administering a therapeutically effective amount of the immunologic composition of claim 4, to the subject in need thereof,
wherein the immunologic composition or the peptide or the antibody or antigen binding fragment thereof effectively treats or prevents SARS-CoV-2 infection in the subject.

23. (canceled)

24. The method of claim 1, wherein the peptide comprises an amino acid sequence selected from SEQ ID NOS: 35 to 57.


SEQUENCE LISTING (57 sequences)

SEQ ID NO: 1 - protein, 75 aa, Severe acute respiratory syndrome coronavirus 2
LKYNENGTITDAVDCALDPLSETKCTLKSFTVEKGIYQTSNFRVQPTESIVRFPNITNLCPFGEVFNATRFASVY

SEQ ID NO: 2 - protein, 65 aa, Severe acute respiratory syndrome coronavirus 2
DITPCSFGGVSVITPGTNTSNQVAVLYQGVNCTEVPVAIHADQLTPTWRVYSTGSNVFQTRAGCL

SEQ ID NO: 3 - protein, 89 aa, Severe acute respiratory syndrome coronavirus 2
FLLKYNENGTITDAVDCALDPLSETKCTLKSFTVEKGIYQTSNFRVQPTESIVRFPNITNLCPFGEVFNATRFASVYAWNRKRISNCVA

SEQ ID NO: 4 - protein, 292 aa, Severe acute respiratory syndrome coronavirus 2
DHWADKYWRAAMSWHRWWPDYQPDYWHDYYYRWHQMQERQMNRWQERWWHRQAYSSPQQTESWRIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 292 aa, Severe acute respiratory syndrome coronavirus 2
DHWADKYWRAAMSWHRWWPDYQPDYWHDYYYRWHQMQERQMNRWQERWWHRQAYSSPQLTPTWRVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 292 aa, Severe acute respiratory syndrome coronavirus 2
DHWADKYWRAAMSWHRWWPDYQPDYWHDYYYRWHQMQERQMNRWQERWWHRQAGSTPQLTPTWRVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 65 aa, Severe acute respiratory syndrome coronavirus 2
DHWADKYWRAAMSWHRWWPDYQPDYWHDYYYRWHQMQERQMNRWQERWWHRQAYSSPQQTESWRI

SEQ ID NO: 8 - protein, 53 aa, Severe acute respiratory syndrome coronavirus 2
SWHRWWPDYQPDYWHDYYYRWHQMQERQMNRWQERWWHRQAYSSPQLTPTWRV

SEQ ID NO: 9 - protein, 65 aa, Severe acute respiratory syndrome coronavirus 2
DHWADKYWRAAMSWHRWWPDYQPDYWHDYYYRWHQMQERQMNRWQERWWHRQAGSTPQLTPTWRV

SEQ ID NO: 10 - protein, 136 aa, Severe acute respiratory syndrome coronavirus 2
FLLKYNENGTITDAVDCALDPLSETKCTLKSFTVEKGIYQTSNFRVQPTESIVRFPNITNLCPFGSNNLDSKVGGNYNYLYRLFRKSNLKPFERDISTEIYQAGSTPCNGVEGFNCYFPLQSYGFQPTNGVGYQPY

SEQ ID NO: 11 - protein, 160 aa, Severe acute respiratory syndrome coronavirus 2
FLLKYNENGTITDAVDCALDPLSETKCTLKSFTVEKGIYQTSNFRVQPTESIVRFPNITNLCPFGEVFNATRFASVYAWNRKRISNCVASNNLDSKVGGNYNYLYRLFRKSNLKPFERDISTEIYQAGSTPCNGVEGFNCYFPLQSYGFQPTNGVGYQPY

SEQ ID NO: 12 - protein, 136 aa, Severe acute respiratory syndrome coronavirus 2
DHWADKYWRAAMSWHRWWPDYQPDYWHDYYYRWHQMQERQMNRWQERWWHRQAYSSPQLTPTWRVSNNLDSKVGGNYNYLYRLFRKSNLKPFERDISTEIYQAGSTPCNGVEGFNCYFPLQSYGFQPTNGVGYQPY

SEQ ID NO: 13 - protein, 136 aa, Severe acute respiratory syndrome coronavirus 2
DHWADKYWRAAMSWHRWWPDYQPDYWHDYYYRWHQMQERQMNRWQERWWHRQAGSTPQLTPTWRVSNNLDSKVGGNYNYLYRLFRKSNLKPFERDISTEIYQAGSTPCNGVEGFNCYFPLQSYGFQPTNGVGYQPY

SEQ ID NO: 14 - protein, 284 aa, Severe acute respiratory syndrome coronavirus 2
DHWADKYWRAAMSWHRWWPDYQPDYWHDYYYRWHQMQERQMNRWQERWWHRQAYSSPQQTESWRIRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 15 - protein, 284 aa, Severe acute respiratory syndrome coronavirus 2
DHWADKYWRAAMSWHRWWPDYQPDYWHDYYYRWHQMQERQMNRWQERWWHRQAYSSPQLTPTWRVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 16 - protein, 284 aa, Severe acute respiratory syndrome coronavirus 2
DHWADKYWRAAMSWHRWWPDYQPDYWHDYYYRWHQMQERQMNRWQERWWHRQAGSTPQLTPTWRVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 17 - protein, 65 aa, Severe acute respiratory syndrome coronavirus 2
DKQADKWWDYATTWHDQQPDWQPDYQKDWYWRQHSTMERQTGDWSEDQQHDQYYRTHQMRERQRI

SEQ ID NO: 18 - protein, 136 aa, Severe acute respiratory syndrome coronavirus 2
DKQADKWWDYATTWHDQQPDWQPDYQKDWYWRQHSTMERQTGDWSEDQQHDQYYRTHQMRERQRISNNLDSKVGGNYNYLYRLFRKSNLKPFERDISTEIYQAGSTPCNGVEGFNCYFPLQSYGFQPTNGVGYQPY

SEQ ID NO: 19 - protein, 284 aa, Severe acute respiratory syndrome coronavirus 2
DKQADKWWDYATTWHDQQPDWQPDYQKDWYWRQHSTMERQTGDWSEDQQHDQYYRTHQMRERQRIRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 20 - protein, 363 aa, Severe acute respiratory syndrome coronavirus 2
DKQADKWWDYATTWHDQQPDWQPDYQKDWYWRQHSTMERQTGDWSEDQQHDQYYRTHQMRERQRISNNLDSKVGGNYNYLYRLFRKSNLKPFERDISTEIYQAGSTPCNGVEGFNCYFPLQSYGFQPTNGVGYQPYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 338 aa, Severe acute respiratory syndrome coronavirus 2
SQCVNDTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLADHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASV

SEQ ID NO: 22 - protein, 335 aa, Severe acute respiratory syndrome coronavirus 2
YAWNRKRISNCVADYSVLYNSASFSTFKCYGVSPTKLNDLCFTNVYAISFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAR

SEQ ID NO: 23 - protein, 155 aa, Severe acute respiratory syndrome coronavirus 2
GKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNL

SEQ ID NO: 24 - protein, 473 aa, Severe acute respiratory syndrome coronavirus 2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSDLDVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITLAVDCALDPLSETKCTLKSFTVEKGIYQTSNFRVQPTEVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYF

SEQ ID NO: 25 - protein, 171 aa, Severe acute respiratory syndrome coronavirus 2
PLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 26 - protein, 644 aa, Severe acute respiratory syndrome coronavirus 2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSDLDVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITLAVDCALDPLSETKCTLKSFTVEKGIYQTSNFRVQPTIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 27 - protein, 644 aa, Severe acute respiratory syndrome coronavirus 2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 28 - protein, 644 aa, Severe acute respiratory syndrome coronavirus 2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTEVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 29 - protein, 646 aa, Severe acute respiratory syndrome coronavirus 2
VNLTTRTQDPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKDYSKHTPINLVRDLPQGFSALEPLVDLPIGINITRFQTLDALHRSYLTPGDSSSGWTAGAAAYYVGYLQPRTFLDKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 30 - protein, 645 aa, Severe acute respiratory syndrome coronavirus 2
VNLTTRTQLPPAYTNSFTRGVYYPDKVFRSSVLHSTQLLFLPFFSNVTWFHAIHVSGTNGTKRFDNPVLPFNLGVYFASTEKSNIIRGWIFGTTLDSKTQSLLIVNNATNVVIKVCEFQFCNDPFLGVYYHKNNKSMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 31 - protein, 646 aa, Severe acute respiratory syndrome coronavirus 2
VNLTTRTQDPPAYTNSFTRGVYYPDKVFRSSVLHSTQDLFLPFFSNVTWFHAIHVSGTNGTKRFDNPVLPFNLGVYFASTEKSNIIRGWIFGTTLDSKTQSDLDVNNATNVVIKVCEFQFCNDPFLGVYYHKNNKSWMESEFRVYSSANNCTFEYVSQPFLMDLEGKQGNFKNLREFVFKNIDGYFKDYSKHTPINLVRDLPQGFSALEPLVDLPIGINITRFQTLDALHRSYLTPGDSSSGWTAGAAAYYVGYLQPRTFLDKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 32 - protein, 646 aa, Severe acute respiratory syndrome coronavirus 2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSDLDVNNATNVVIKVCEFQFCNDPFLGVYYHKNNKSWMESEFRVYSSANNCTFEYVSQPFLMDLEGKQGNFKNLREFVFKNIDGYFKDYSKHTPINLVRDLPQGFSALEPLVDLPIGINITRFQTLDALHRSYLTPGDSSSGWTAGAAAYYVGYLQPRTFLDKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 33 - protein, 646 aa, Severe acute respiratory syndrome coronavirus 2
VNLTLRTQLPPAYTNSFTRGVYYPDKVFRSSVLHSTQLLFLPFFSNVTWFHAIHVSGTNGTKRFDNPVLPFNLGVYFASTEKSNIIRGWIFGTTLDSKTQSDLDVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYE

SEQ ID NO: 34 - protein, 646 aa, Severe acute respiratory syndrome coronavirus 2; identical to SEQ ID NO: 31

SEQ ID NO: 35 - protein, 75 aa, Severe acute respiratory syndrome coronavirus 2; identical to SEQ ID NO: 1

SEQ ID NO: 36 - protein, 65 aa, Severe acute respiratory syndrome coronavirus 2; identical to SEQ ID NO: 2

SEQ ID NO: 37 - protein, 89 aa, Severe acute respiratory syndrome coronavirus 2; identical to SEQ ID NO: 3

SEQ ID NO: 38 - protein, 292 aa, Severe acute respiratory syndrome coronavirus 2; identical to SEQ ID NO: 4

SEQ ID NO: 39 - protein, 292 aa, Severe acute respiratory syndrome coronavirus 2; identical to SEQ ID NO: 5

SEQ ID NO: 40 - protein, 292 aa, Severe acute respiratory syndrome coronavirus 2; identical to SEQ ID NO: 6

SEQ ID NO: 41 - protein, 65 aa, Severe acute respiratory syndrome coronavirus 2; identical to SEQ ID NO: 7

SEQ ID NO: 42 - protein, 53 aa, Severe acute respiratory syndrome coronavirus 2; identical to SEQ ID NO: 8

SEQ ID NO: 43 - protein, 65 aa, Severe acute respiratory syndrome coronavirus 2; identical to SEQ ID NO: 9

SEQ ID NO: 44 - protein, 136 aa, Severe acute respiratory syndrome coronavirus 2; identical to SEQ ID NO: 10

SEQ ID NO: 45 - protein, 160 aa, Severe acute respiratory syndrome coronavirus 2; identical to SEQ ID NO: 11

SEQ ID NO: 46 - protein, 136 aa, Severe acute respiratory syndrome coronavirus 2; identical to SEQ ID NO: 12

SEQ ID NO: 47 - protein, 136 aa, Severe acute respiratory syndrome coronavirus 2; identical to SEQ ID NO: 13

SEQ ID NO: 48 - protein, 284 aa, Severe acute respiratory syndrome coronavirus 2; identical to SEQ ID NO: 14

SEQ ID NO: 49 - protein, 284 aa, Severe acute respiratory syndrome coronavirus 2; identical to SEQ ID NO: 15

SEQ ID NO: 50 - protein, 284 aa, Severe acute respiratory syndrome coronavirus 2; identical to SEQ ID NO: 16

SEQ ID NO: 51 - protein, 65 aa, Severe acute respiratory syndrome coronavirus 2; identical to SEQ ID NO: 17

SEQ ID NO: 52 - protein, 136 aa, Severe acute respiratory syndrome coronavirus 2; identical to SEQ ID NO: 18

SEQ ID NO: 53 - protein, 284 aa, Severe acute respiratory syndrome coronavirus 2; identical to SEQ ID NO: 19

SEQ ID NO: 54 - protein, 363 aa, Severe acute respiratory syndrome coronavirus 2; identical to SEQ ID NO: 20

SEQ ID NO: 55 - protein, 673 aa, Severe acute respiratory syndrome coronavirus 2
SQCVNDTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLADHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYAISFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAR

SEQ ID NO: 56 - protein, 155 aa, Severe acute respiratory syndrome coronavirus 2; identical to SEQ ID NO: 23

SEQ ID NO: 57 - protein, 155 aa, Severe acute respiratory syndrome coronavirus 2
GKQGNFKNLREFVFKNIDGYFKIYSKHTPINLDRILPQGFSALEPLVDLPIGINITRFQTLLADHRSYLTPGDSSSGWTAGAAAYYDGYLQPRTFLLKYNENGTITDAVDCALDPLSETKCTLKSFTVEKGIYQTSNFRVQPTESIVRFPNITNL